(19) United States
(12) Patent Application Publication
(10) Pub. No.: US 2016/0106341 A1
(43) Pub. Date: Apr. 21, 2016

(54) DETERMINING RESPIRATORY PARAMETERS

(71) Applicant: PULMONE ADVANCED MEDICAL DEVICES, LTD., Ra'anana (IL)

(72) Inventors: Ori Adam, Rechovot (IL); Adam Laprad, San Francisco, CA (US); Inon Cohen, Petach Tiqva (IL); Zachi Peles, Tel Aviv (IL); Julian Solway, Glencoe (IL); Jeffrey J. Fredberg, Chestnut Hill, MA (US)

(21) Appl. No.: 14/780,950

(22) PCT Filed: Mar. 28, 2014
(86) PCT No.: PCT/US2014/032186

§ 371 Date: Sep. 28, 2015

Related U.S. Application Data
(60) Provisional application No. 61/844,182, filed on Jul. 9, 2013.

(51) Int. Cl. A61B 5/097; A61B 5/091; A61B 5/00; A61B 5/08; A61B 5/085; A61B 5/087; A61B 6/03

(52) U.S. Cl. CPC A61B 5/097; A61B 5/087; A61B 5/091; A61B 6/032; A61B 5/0806; A61B 5/085; A61B 5/7267; A61B 5/7278; A61B 2505/03; A61B 2503/42; A61B 2503/12; A61B 2505/07

(57) ABSTRACT

A pulmonary measurement system includes a pulmonary measurement device that includes a mouthpiece with an airflow path and a sensor positioned in the airflow path; and a controller communicably coupled to the sensor. The controller includes a processor and instructions stored in memory and is operable to execute the instructions with the processor to perform operations including identifying a measurement from the sensor; identifying a particular equation stored in the memory, the particular equation developed using data analytics and including an input parameter that is based on the identified measurement; and based on the identified measurement and the particular equation, determining a value of absolute lung volume.

### CLAIM OF PRIORITY

This application claims priority to U.S. Provisional Application No. 61/844,182 filed on Jul. 9, 2013, the entire contents of which are hereby incorporated by reference.

### TECHNICAL BACKGROUND

This disclosure relates to methods for estimating (e.g., calculating) respiratory health parameters and/or to a method for estimating (e.g., calculating) pulmonary function parameters using measured and/or known input parameters.

### BACKGROUND

Absolute lung volume is a key parameter in pulmonary physiology and diagnosis, but it is not easy to measure in a live individual. It is relatively straightforward to measure the volume of air that is exhaled from a subject's mouth, but at the end of complete exhalation, a significant amount of air is left in the lungs because the mechanical properties of the lungs and chest wall, including the ribs, do not allow the lungs to collapse completely. The gas remaining in the lungs at the end of a complete exhalation is termed the Residual Volume (RV) and may be significantly increased or decreased in disease. The total volume of gas in the lungs at the end of a maximal inspiration is termed the Total Lung Capacity (TLC). The TLC includes the RV plus the maximum amount of gas that can be inhaled or exhaled, which is termed the Vital Capacity (VC). However, during normal breathing, the subject does not empty the lungs down to RV or inflate the lungs to TLC. The amount of gas remaining in the lungs at the end of a normal breath, as distinct from a complete exhalation, is termed the Functional Residual Capacity (FRC) or Thoracic Gas Volume (TGV), depending upon the manner in which it is measured. For simplicity, when this volume is measured by inert gas dilution techniques, it will be termed FRC, and when it is measured by barometric techniques involving gas compression, it will be termed TGV, as described in this application.

In order to determine the total volumes of gas in the lungs at TLC, FRC, TGV, or RV, indirect methods may be used since it is impossible to completely exhale all of the gas from the lungs. Acceptable techniques for measuring lung volumes in humans include, for example: (1) Whole Body Plethysmography, in which a subject makes respiratory efforts against an obstruction within a gas tight chamber and the changes in pressure on the patient side of the obstruction can be related to the changes in pressure in the chamber through Boyle's law to calculate TGV; (2) Multi-breath Helium Gas Dilution involving the dilution of a known concentration and volume of Helium by the gas in the lungs of a subject; (3) Nitrogen wash-out, in which upon the expiration of a known gas volume with 100% oxygen, the time required to resume normal atmospheric nitrogen concentrations is used to estimate lung volume; (4) Computerized Tomography, in which three-dimensional imaging of the lungs is used to estimate lung volume; and (5) Chest Radiography, in which lung volume is estimated from chest radiography images. The most commonly used techniques, however, are gas dilution and whole body plethysmography.

While the above-mentioned techniques for measuring lung volumes in humans are considered acceptable, such techniques may produce undesired measurement inaccuracies, may require complicated and/or expensive equipment, or may be difficult to perform. For example, gas dilution involves the dilution of a known concentration and volume of inert gas by the gas in the lungs of a subject and is critically dependent on complete mixing of the marker gas and lung gas. In subjects with poor gas mixing due to disease, this technique is very inaccurate and generally underestimates the true FRC. Whole body plethysmography is generally believed to accurately measure TGV even in sick subjects, but requires complicated and expensive equipment and is difficult to perform. Several studies, however, have shown that whole body plethysmography may overestimate lung volumes in severely obstructed patients.

Once FRC (e.g., determined by gas dilution) or TGV (e.g., determined by whole body plethysmography) is calculated, measurement by spirometry of the extra volume of gas that can be exhaled from the end of a normal exhalation (Expiratory Reserve Volume, ERV) and the extra volume that can be inhaled from the end of a normal exhalation (Inspiratory Capacity, IC) allows the calculation of TLC and RV.

These three important indicators (TLC, RV, and FRC or TGV) are mutually related through the following formulas: RV=FRC−ERV, TLC=FRC+IC, and TLC=RV+ERV+IC=RV+VC.

If FRC is determined by gas dilution and TGV is determined by a barometric method, then the difference between TGV and FRC is a measure, albeit approximate, of the volume of poorly ventilated or “trapped gas” in the lungs.

In healthy subjects, TGV and FRC is approximately equal as there is little or no trapped gas, and hence, for practical matters, in this disclosure the term TGV shall be used as a synonym for FRC. In summary, determination of absolute lung volume (e.g., TLC, TGV, and RV) may be central for the evaluation of lung function but is not easily determined from existing technologies.

### SUMMARY

In one general implementation, a pulmonary measurement system includes a pulmonary measurement device that includes a mouthpiece with an airflow path and a sensor positioned in the airflow path; and a controller communicably coupled to the sensor. The controller includes a processor and instructions stored in memory and is operable to execute the instructions with the processor to perform operations including identifying a measurement from the sensor; identifying a particular equation stored in the memory, the particular equation developed using data analytics and including an input parameter that is based on the identified measurement; and based on the identified measurement and the particular equation, determining a value of absolute lung volume.

In a first aspect combinable with the general implementation, the sensor includes at least one of an airflow sensor or a pressure sensor.

In a second aspect combinable with any of the previous aspects, the controller is operable to execute the instructions with the processor to perform further operations including: determining the input parameter to the particular equation based on the measurement; and calculating the value of absolute lung volume based on the input parameter.

In a third aspect combinable with any of the previous aspects, the input parameter includes a parameter related to respiratory function, respiratory mechanics, respiratory health, or general health.

In a fourth aspect combinable with any of the previous aspects, the input parameter includes at least one of an airway opening pressure, a derivative of the airway opening pressure, an integral of the airway opening pressure, an airway opening flowrate, a derivative of the airway opening flowrate, an integral of the airway opening flowrate, a parameter derivable from forced spirometry, a parameter derivable from slow spirometry, a mechanical impedance, a parameter derivable from forced oscillations, a parameter derivable from impulse oscillometry, a time constant of a pressure decay or rise, or a time constant of a flowrate decay or rise.

In a fifth aspect combinable with any of the previous aspects, the pulmonary measurement device includes one of: (a) a spirometer; (b) a forced oscillation device; (c) an advanced flow interruption device; (d) a flow interruption device; (e) a combination spirometer-flow interruption device; or (f) a combination device of two or more of (a)-(e).

A sixth aspect combinable with any of the previous aspects further includes a handheld housing that at least partially encloses or couples to the pulmonary measurement device and the controller.

In a seventh aspect combinable with any of the previous aspects, identifying a particular equation includes identifying the particular equation from a plurality of equations that are stored in the memory.

In an eighth aspect combinable with any of the previous aspects, the controller is operable to execute the instructions with the processor to perform further operations including identifying a second particular equation of the plurality of equations that are stored in the memory, the second particular equation developed using data analytics and including a second input parameter that is based on the identified measurement; and based on the identified measurement and the second particular equation, determining at least one of total lung capacity (TLC), functional residual capacity (FRC), thoracic gas volume (TGV), residual volume (RV), diffusing capacity of the lung for carbon monoxide (DLCO), airway resistance, lung elasticity, or lung tissue compliance.

In a ninth aspect combinable with any of the previous aspects, the controller is operable to execute the instructions with the processor to perform further operations including identifying a third particular equation of the plurality of equations that are stored in the memory, the third particular equation developed using data analytics and including a third input parameter that is based on the identified measurement; and based on the identified measurement and the third particular equation, determining at least one qualitative indicator of respiratory health.

In a tenth aspect combinable with any of the previous aspects, the at least one qualitative indicator of respiratory health includes a diagnosis of: health, obstructive respiratory disease, restrictive respiratory disease, mixed defect, pulmonary vascular disorder, chest wall disorder, neuromuscular disorder, interstitial lung disease, pneumonitis, asthma, chronic bronchitis, or emphysema.

In an eleventh aspect combinable with any of the previous aspects, the particular equation is derived from a training population that includes a plurality of healthy subjects.

In a twelfth aspect combinable with any of the previous aspects, the particular equation is derived from a training population that further includes a plurality of unhealthy subjects.

In a thirteenth aspect combinable with any of the previous aspects, each of the plurality of unhealthy subjects has one or more respiratory diseases.

In a fourteenth aspect combinable with any of the previous aspects, the particular equation includes a constant that is calculated based on a respiratory measurement technique performed on the training population.

In a fifteenth aspect combinable with any of the previous aspects, the respiratory measurement technique includes at least one of body plethysmography, helium dilution, or thoracic computed tomography (CT) imaging.

In a sixteenth aspect combinable with any of the previous aspects, the training population includes historical or public data.

In a seventeenth aspect combinable with any of the previous aspects, the training population includes a first portion and a second portion, each of the first and second portions defined by a classifier.

In an eighteenth aspect combinable with any of the previous aspects, the classifier includes an anthropomorphic or a spirometric classifier, and the controller is operable to execute the instructions with the processor to perform further operations including selecting the particular equation based, at least in part, on the classifier.

In a nineteenth aspect combinable with any of the previous aspects, the respiratory measurement occurs in one of an intensive care unit, a pulmonary function testing laboratory, a physician's office, a community/work screening, or a home setting.

In a twentieth aspect combinable with any of the previous aspects, the particular equation includes a linear equation or a non-linear equation.

In a twenty-first aspect combinable with any of the previous aspects, the particular equation is derived from a regression analysis.

In a twenty-second aspect combinable with any of the previous aspects, the controller is operable to execute the instructions with the processor to perform further operations including updating at least one of the plurality of equations that are stored in the memory based on at least one of a time duration or an adjustment to the data analytics.

In a twenty-third aspect combinable with any of the previous aspects, the adjustment to the data analytics includes an increase in a number of subjects of a training population used to derive the plurality of equations.

In another general implementation, a computer-implemented method to determine absolute lung volume includes identifying a particular equation that is developed using data analytics and includes an input parameter; identifying a respiratory measurement of a patient with a pulmonary measurement device, the input parameter based on the identified respiratory measurement; and based on the respiratory measurement of the patient and the particular equation, determining the absolute lung volume of the patient.

In a first aspect combinable with the general implementation, identifying a particular equation includes identifying the particular equation from a plurality of equations.

A second aspect combinable with any of the previous aspects further includes identifying a second particular equation of the plurality of equations that is developed using data analytics and includes a second input parameter; and based on the respiratory measurement of the patient and the second particular equation, determining at least one of total lung capacity (TLC), functional residual capacity (FRC), thoracic gas volume (TGV), residual volume (RV), diffusing capacity of the lung for carbon monoxide (DLCO), airway resistance, or lung tissue compliance.

In a third aspect combinable with any of the previous aspects, the particular equation is determined based on a training population using clinical data.

In a fourth aspect combinable with any of the previous aspects, the training population includes healthy subjects and unhealthy subjects.

In a fifth aspect combinable with any of the previous aspects, each of the unhealthy subjects have one or more respiratory diseases.

A sixth aspect combinable with any of the previous aspects further includes generating the data analytics by measuring an absolute lung volume value of each subject of the training population using a respiratory testing technique.

A seventh aspect combinable with any of the previous aspects further includes obtaining the at least one respiratory measurement with the pulmonary measurement device.

In an eighth aspect combinable with any of the previous aspects, the pulmonary measurement device includes one of: (a) a spirometer; (b) a forced oscillation device; (c) an advanced flow interruption device; (d) a flow interruption device; (e) a combination spirometer-flow interruption device; or (f) a combination device of two or more of (a)-(e).

In a ninth aspect combinable with any of the previous aspects, the input parameter includes a parameter related to respiratory function, respiratory mechanics, respiratory health, or general health.

In a tenth aspect combinable with any of the previous aspects, the input parameter is selected based on a known correlation between the input parameter and absolute lung volume.

In an eleventh aspect combinable with any of the previous aspects, the input parameter includes at least one of an airway opening pressure, a derivative of the airway opening pressure, an integral of the airway opening pressure, an airway opening flowrate, a derivative of the airway opening flowrate, an integral of the airway opening flowrate, a parameter derivable from forced spirometry, a parameter derivable from slow spirometry, a mechanical impedance, a parameter derivable from forced oscillations, a parameter derivable from impulse oscillometry, a time constant of a pressure decay or rise, or a time constant of a flowrate decay or rise.

In a twelfth aspect combinable with any of the previous aspects, the respiratory testing technique includes body plethysmography, helium dilution, or thoracic computed tomography (CT) imaging.

In a thirteenth aspect combinable with any of the previous aspects, the particular equation includes a linear equation.

In another general implementation, a method of estimating a respiratory parameter of a human subject includes taking a direct measurement of a respiratory parameter in a plurality of test subjects, the plurality of test subjects including healthy subjects and unhealthy subjects; taking a measurement of one or more input parameters of the plurality of test subjects; and determining, with the direct measurements of the respiratory parameter and the measurements of one or more input parameters, an equation that includes at least a portion of the input parameters as inputs and the respiratory parameter as an output.

In a first aspect combinable with the general implementation, each of input parameters is associated with the respiratory parameter.

In a second aspect combinable with any of the previous aspects, taking a direct measurement of a respiratory parameter in a plurality of test subjects is performed with at least one of a whole body plethysmography technique, a helium dilution technique, a thoracic computed tomography (CT) imaging technique, a nitrogen washout, a nitrogen recovery, or a chest radiography.

In a third aspect combinable with any of the previous aspects, taking a measurement of one or more input parameters of the plurality of test subjects is performed with at least one of a pulmonary measurement device, a spirometer, a flow interruption device, an advanced flow interruption device, a forced oscillation or impulse oscillometry technique, or an anthropomorphic device.

In a fourth aspect combinable with any of the previous aspects, at least one of the one or more input parameters includes a relative lung volume or a lung flow rate.

In a fifth aspect combinable with any of the previous aspects, the relative lung volume comprises at least one of: forced expiratory volume in one second (FEV₁), a ratio of forced expiratory volume in one second to forced vital capacity (FEV₁/FVC), inspiratory capacity (IC), or vital capacity (VC).

In a sixth aspect combinable with any of the previous aspects, at least one of the one or more input parameters includes at least one of an airway opening pressure, a derivative of the airway opening pressure, an integral of the airway opening pressure, an airway opening flowrate, a derivative of the airway opening flowrate, an integral of the airway opening flowrate, a parameter derivable from forced spirometry, a parameter derivable from slow spirometry, a mechanical impedance, a parameter derivable from forced oscillations, a parameter derivable from impulse oscillometry, a time constant of a pressure decay or rise, or a time constant of a flowrate decay or rise.

In a seventh aspect combinable with any of the previous aspects, at least one of the one or more input parameters includes a respiratory mechanics value including respiratory system resistance (Rᵣₛ) or respiratory system elastance (Eᵣₛ).

In an eighth aspect combinable with any of the previous aspects, at least one of the one or more input parameters includes anthropomorphic information that includes one or more of patient sex, patient height, patient weight, or patient body mass index.

In a ninth aspect combinable with any of the previous aspects, the respiratory parameter comprises at least one of: total lung capacity (TLC), thoracic gas volume (TGV), residual volume (RV), or functional residual capacity (FRC).

A tenth aspect combinable with any of the previous aspects further includes taking a measurement of one or more input parameters of a human subject with a pulmonary measurement device; and based on the measurement of one or more input parameters of the human subject and the equation, estimating a value of the respiratory parameter of the human subject with the pulmonary measurement device.

Various embodiments disclosed herein may include one or more of the following features. For example, various embodiments may implement handheld or desktop pulmonary measurement devices to obtain clinically accurate absolute lung volumes (ALVs) based on measures of respiratory mechanics and lung function at the mouth (e.g., without directly measuring ALV). Further, various embodiments may implement handheld or desktop pulmonary measurement devices to obtain clinically accurate ALV values based, at least in part, on an equation that is deduced from physiological or physical considerations. Various embodiments may also implement handheld or desktop pulmonary measurement devices using an equation that is developed using data analytics approaches and is based on a training population of healthy as well as diseased patients. Various embodiments may obtain clinically accurate absolute lung volumes for general populations of healthy as well as diseased patients.

These general and specific embodiments may be implemented using a device, system or method, or any combinations of devices, systems, or methods. The details of one or more embodiments are set forth in the accompanying drawings and the description below. Other features, objects, and advantages will be apparent from the description and drawings, and from the claims.

### DESCRIPTION OF DRAWINGS

FIG. 1 illustrates an example pulmonary measurement device configured to perform one or more processes and operations in accordance with the present disclosure;

FIG. 2 illustrates another example pulmonary measurement device configured to perform one or more processes and operations in accordance with the present disclosure;

FIG. 3 illustrates another example pulmonary measurement device configured to perform one or more processes and operations in accordance with the present disclosure;

FIG. 4 illustrates another example pulmonary measurement device configured to perform one or more processes and operations in accordance with the present disclosure;

FIG. 5 illustrates another example pulmonary measurement device configured to perform one or more processes and operations in accordance with the present disclosure;

FIG. 6 is a flowchart of an example method for calculating a desired respiratory parameter on any healthy or unhealthy subject with a pulmonary measurement device that executes a mathematical equation that estimates the desired parameter;

FIG. 7 is a flowchart of an example method for generating a mathematical equation that estimates a desired output parameter;

FIG. 8 is a block diagram showing the relationship among one or more input parameters, an output parameter, and an equation generated using the example method of FIG. 7;

FIGS. 9A-9P illustrate a number of charts that show a relationship between total lung capacity (TLC) estimated using an equation (TLC_{equation}) on a pulmonary measurement device as shown in FIG. 3 and TLC measured using body plethysmography (TLC_{PLETH});

FIG. 10A-10H illustrates a number of charts that show a relationship between TLC estimated using an equation (TLC_{equation}) on a pulmonary measurement device as shown in FIG. 2 and TLC measured using body plethysmography (TLC_{PLETH}); and

FIG. 11A-11H illustrates a number of charts that show a relationship between TLC estimated using an equation (TLC_{equation}) on a pulmonary measurement device as shown in FIG. 1 and TLC measured using body plethysmography (TLC_{PLETH}).

### DETAILED DESCRIPTION

This disclosure relates to methods for measuring respiratory parameters and, more particularly, to a method for estimating (e.g., calculating) pulmonary function parameters using measured and/or known input parameters.

Absolute lung volume (ALV) is one example of a pulmonary function parameter and is a general term that is used to encompass individual absolute lung volume compartments, including TLC, FRC, TGV, and RV. ALV is a key parameter in pulmonary physiology and diagnosis, but it is not easy to measure in the live individual. While conventional techniques for measuring absolute lung volumes in humans are considered acceptable in many cases, such techniques may produce undesired measurement inaccuracies, may require complicated and/or expensive equipment, or may be difficult to perform.

Devices to measure ALV must have a clinically acceptable accuracy and precision, especially for subjects with respiratory diseases. Clinically acceptable error limits are defined by American Thoracic Society standards as well as by the scientific literature that includes clinical data on devices that measure ALV. For example, certain industry standards require three ALV measurements to agree within 5% for any technique or device that measures ALV, such as body plethysmography and gas dilution techniques. In addition, body plethysmography is widely regarded in the industry as the gold standard device to measure ALV, and alternative devices that measure ALV should produce ALVs that have a basic level of agreement with body plethysmographic measurements. For instance, helium dilution and computed tomography (CT) imaging are two alternative techniques to measure ALV and are based on different physical principles than body plethysmography. Coefficient of variation (CV) is one metric to describe agreement and it encompasses both accuracy and precision of a test method (e.g., helium dilution of CT imaging) compared to a reference method (body plethysmography). An analysis of data from comparative studies within the scientific literature show that the CV of TLC measured by CT imaging is 15.6% compared to body plethysmography and the CV of TLC measured by helium dilution is 18.9% compared to body plethysmography.

While many simple hand-held conventional pulmonary function devices can be used to determine parameters such as those related to spirometry and respiratory mechanics, such devices are not capable of determining ALV during normal operation. In some examples, hand-held devices (e.g., hand-held spirometers) are used to measure the volume of gas exhaled from a subject's mouth during a forced expiration. A residual volume (RV) of gas remains in the lungs at the end of a complete exhalation because the mechanical properties of the respiratory system do not allow the lungs to collapse completely. Therefore, spirometers cannot measure TGV. Rather, spirometers measure relative changes in lung volumes, also known as volume differentials (e.g., vital capacity (VC)), as well as volumes of gas inhaled or exhaled during a given period of time (e.g., forced expiratory volume in one second (FEV₁)).

In some examples, devices (e.g., respiratory mechanics devices) are used to determine various mechanical properties of the respiratory system by measuring mechanical impedances at one or more frequencies. For example, respiratory mechanics can be measured using a variety of devices and techniques, such as impulse oscillometry (IOS), the forced oscillation technique (FOT), and flow interruption (FI). Respiratory mechanics devices have been shown to be incapable of accurately estimating ALV, which is typical represented in mechanical terms as thoracic gas compliance (C_{g}=the ALV divided by the alveolar pressure). Instead, these devices can accurately measure mechanical properties such as respiratory system resistance (Rᵣₛ) (and/or its inverse, respiratory system elastance (Eᵣₛ), respiratory system compliance (Cᵣₛ), airway resistance (R_{aw}), lung tissue compliance (Cₜᵢₛₛ), lung tissue resistance (Rₜᵢₛₛ), chest wall compliance (C_{cw}), and chest wall resistance (R_{cw}).

Other devices (e.g., flow interruption devices) may also be used to measure respiratory mechanics and pulmonary function parameters. For example, flow interruption devices are used to measure airway resistance during interruption (Rᵢₙₜ). Also, advanced flow interruption devices can allow a subject to breath more comfortably from a closed container of gas during a flow interruption event.

While spirometric devices and respiratory mechanics devices may not measure ALV directly, in some cases, some measurements of pulmonary function parameters obtained using such devices are correlated with ALV in healthy patients. For example, R_{aw} is inversely related to ALV. Additionally, Cₜᵢₛₛ is directly related to ALV. Furthermore, FEV₁ and VC are related to ALV in healthy patients.

Although correlations exist between measurements of pulmonary function parameters and TGV in healthy patients, such correlations alone have not produced a clinically acceptable calculation of ALV as defined by American Thoracic Society standards, especially for subjects with respiratory diseases.

Investigators have also explored more complex methods to determine ALV from handheld or table-top devices that measure spirometry, respiratory mechanics, or respiratory dynamics. However, all approaches have failed to produce a clinically acceptable calculation of ALV. For instance, data obtained from respiratory mechanics measurements, even when extended to a wide range of forcing frequencies, have been shown to be inadequate to infer absolute lung volumes in the individual subject. Similarly, data obtained from forced expiratory maneuvers have been shown to be inadequate. This failure may be attributable in part to the fact that the dynamics of gas distribution within the human lung are complex, and especially so in obstructive lung disease. The complex nature makes it difficult to impossible to develop an equation for ALV from idealized mechanical models of the lung and/or physical principles. In addition, this failure may also be attributable in part to the fact that data interpretation often rests upon fitting respiratory impedance data to idealized mathematical models wherein there exists a wide range of models that fit the data equally well. In other instances, this failure may be the result of fitting data to mathematical equations of a simplistic and/or pre-defined physical form. When this happens, no useful estimate of ALV can be determined.

More importantly, since measurements of ALV may be used by clinicians to diagnose respiratory diseases, track progression of respiratory diseases, and develop treatments, any single method to calculate ALV should produce valid measurements for both healthy and diseased patient populations. In this regard, establishing a mathematical relationship between respiratory mechanics, spirometry, and/or other lung function parameters and ALV that is both clinically accurate and applicable to both healthy and diseased populations is non-trivial. Numerous examples show the non-trivial nature of these relationships. For example, in obstructive airway diseases (e.g., asthma), airway resistance may be elevated, while ALV may either remain constant or increase. In destructive lung diseases (e.g., emphysema), tissue compliance may be elevated compared to tissue compliance of a healthy person, while ALV may be lower or higher compared to ALV of a healthy person. These disease-related differences in the measured parameters of respiratory mechanics and/or lung function should be accounted for in order to accurately estimate ALV from the measured parameters.

In some examples, algorithms may be used to generate a mathematical equation for calculating a particular desired output parameter (e.g., ALV) from one or more input parameters measured using one or more devices (e.g., spirometry and respiratory mechanics devices or other hand-held, table-top, or floor-standing respiratory function devices). The mathematical equation may not be derived entirely from physical principles (e.g., Boyle's law) but instead is determined in part or in whole using data analytics (e.g., data mining) techniques.

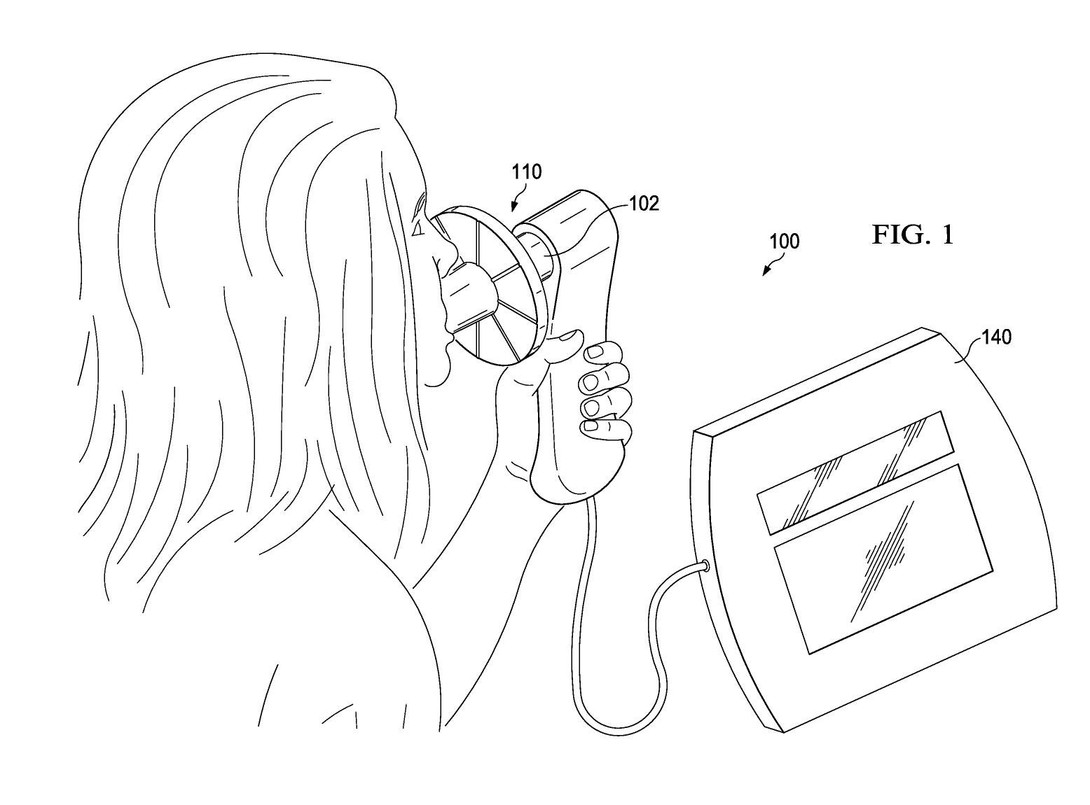

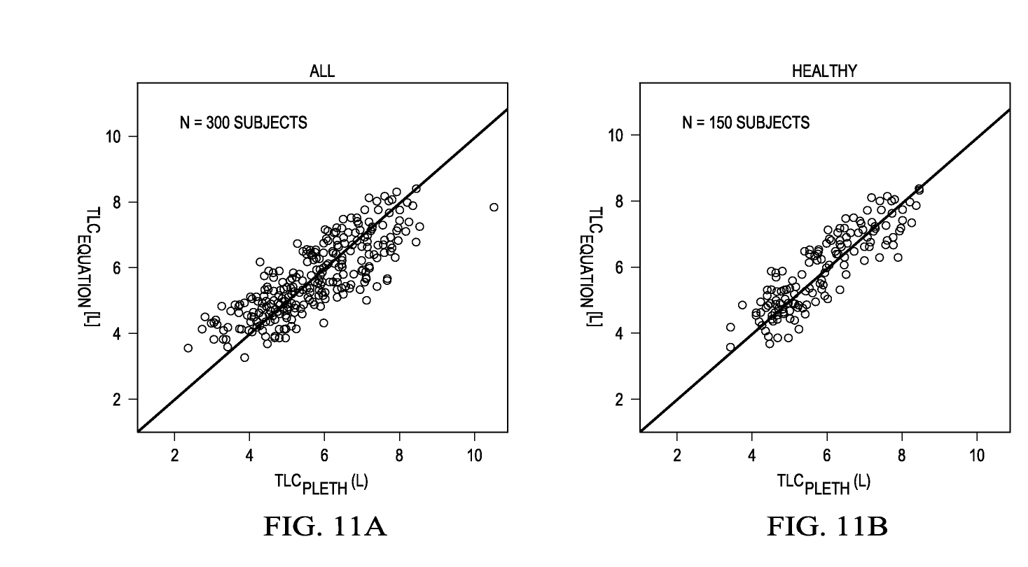

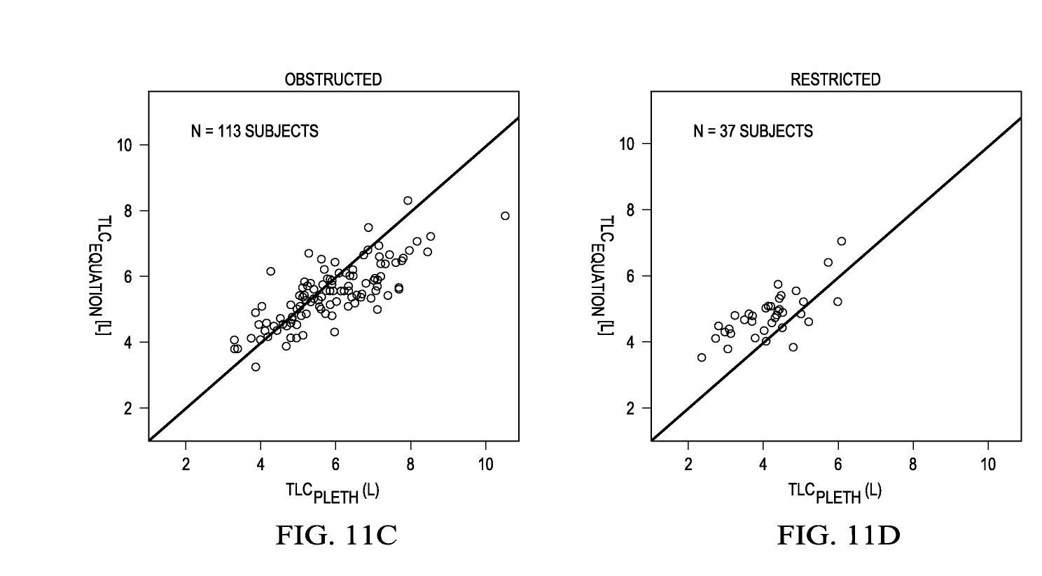

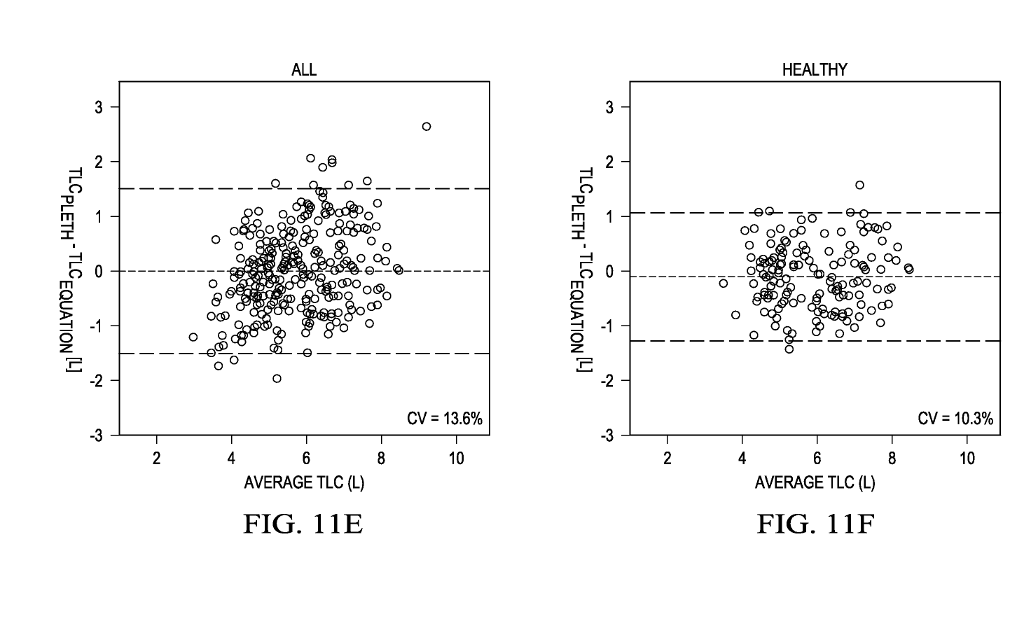

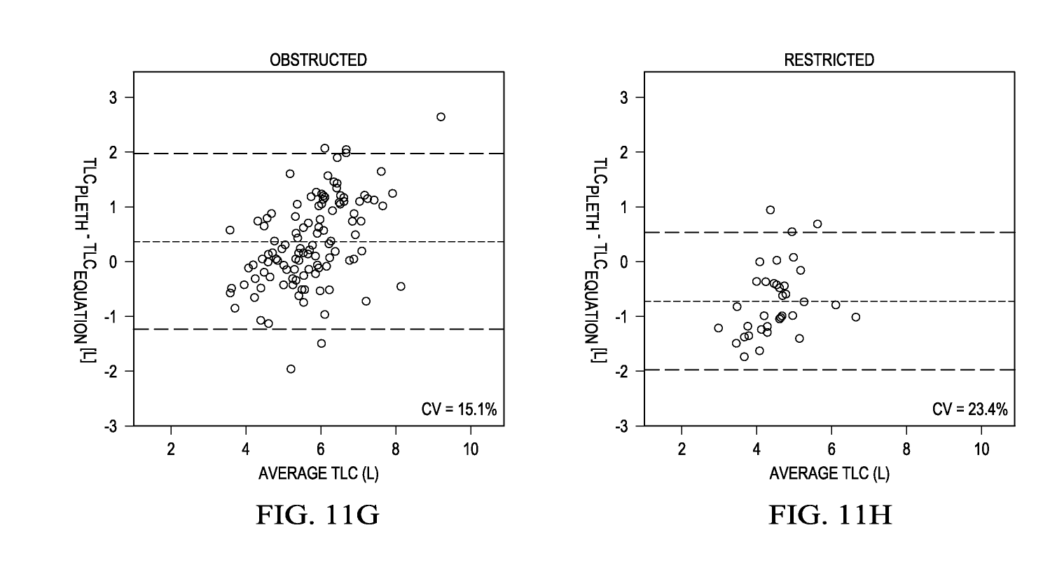

FIG. 1 illustrates an example pulmonary measurement device 100 that is configured to perform one or more processes and operations in accordance with the present disclosure. In some implementations, the device 100 may be a handheld spirometer. In some implementations, the device 100 may be used to perform one or more operations described in the present disclosure, such as for example, one or more steps of method 600. Example data resulting from such operations performed with the device 100 is shown, for example, in FIGS. 11A-11B.

Device 100, as illustrated, includes a bacterial filter 110 coupled to a breathing tube 102 that includes an airflow sensor (e.g., flow sensor, pressure differential sensor, and/or both). The device 100 also includes a controller 140 that is communicably coupled with the breathing tube 102 to receive measurements from, for instance, the airflow sensor, and perform one or more operations on and/or with such measurements.

Generally, for instance, the device 100 gauges lung function by measuring (e.g., with the airflow sensor) a forced expiratory volume, or an amount of air that a patient can expel from his/her lungs within a particular time duration, such as within one second. The device 100 can also measure, with the airflow sensor, a forced vital capacity (FVC), or a total amount of air a patient can expel from his/her lungs. Based, at least in part on these two measurements, a total amount of air that a patient can expel in one breath may be determined by the controller 140. Using these measurements, a medical professional can determine whether a patient's spirometer readings indicate a normal air capacity or an obstructive air capacity, for instance.

Generally, during operation, the patient must breathe in and seal his/her lips around the bacterial filter 110. With the mouth sealed around the filter 110 (e.g., which includes a mouthpiece), the patient blows out air as hard and as fast as possible until there is absolutely no air left in the lungs. The airflow sensor in or part of the breathing tube 102 measures an airflow during exhalation and the resultant measurements may be stored in the controller 140. The patient may perform this maneuver multiple times to achieve an average measurement.

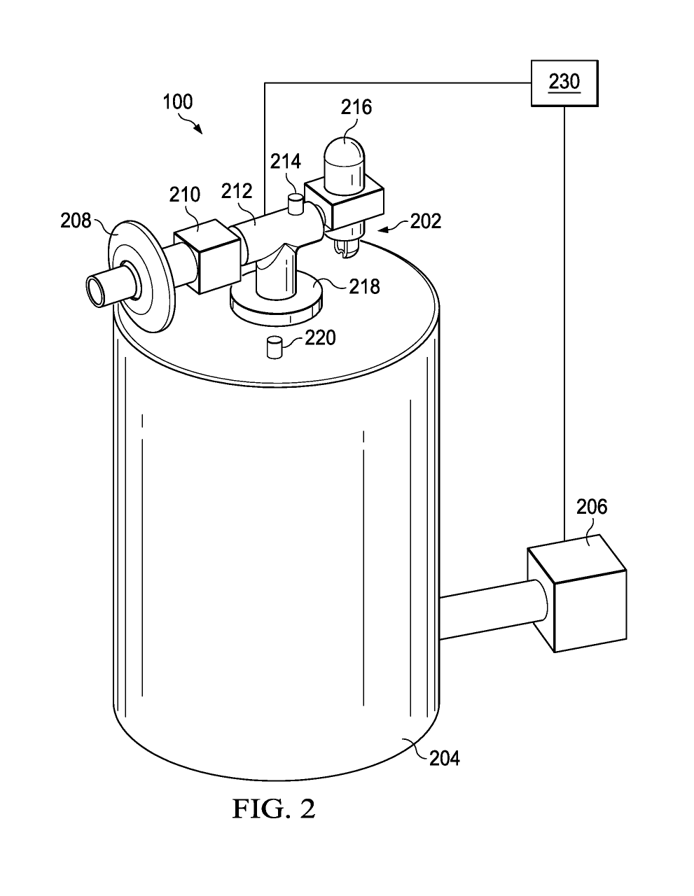

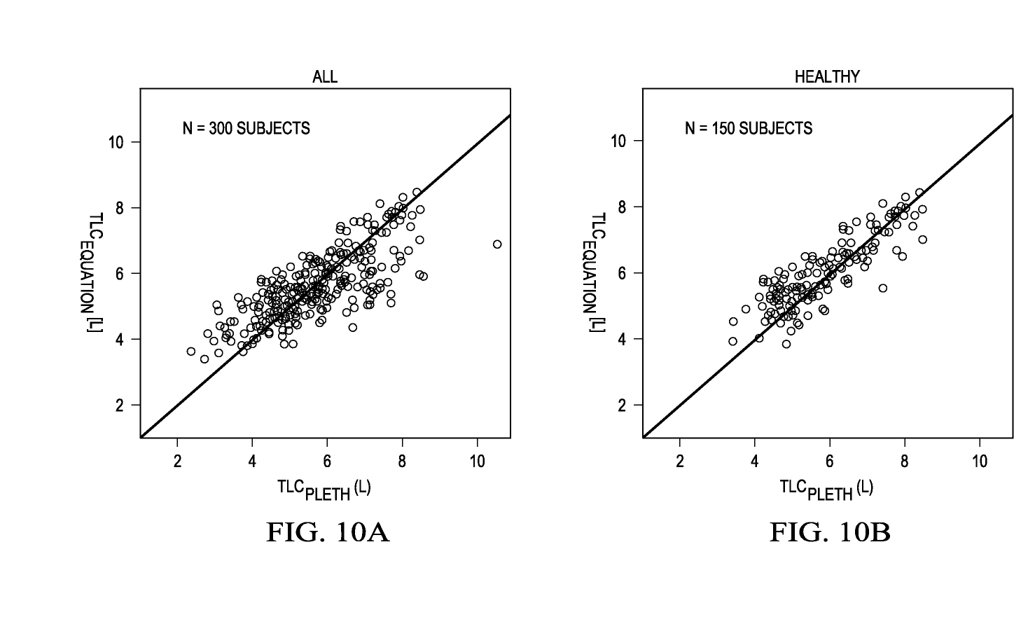

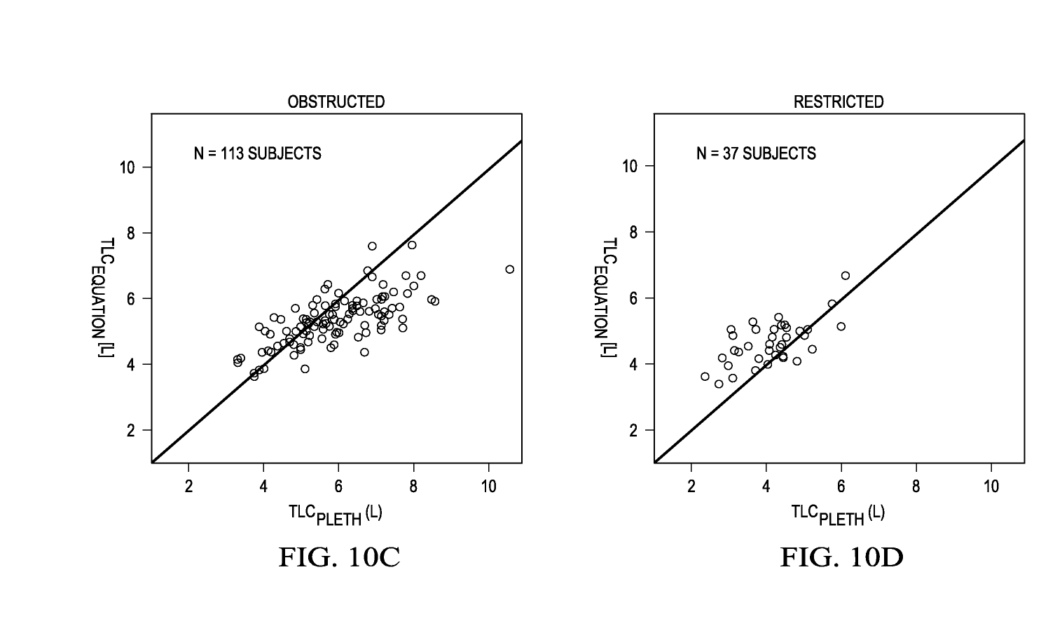

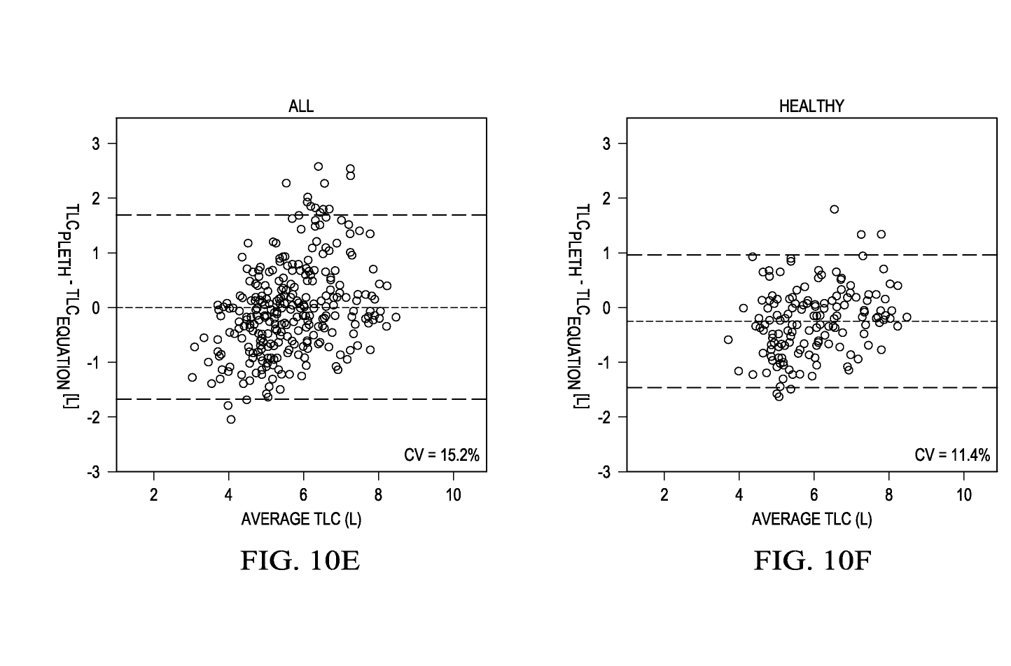

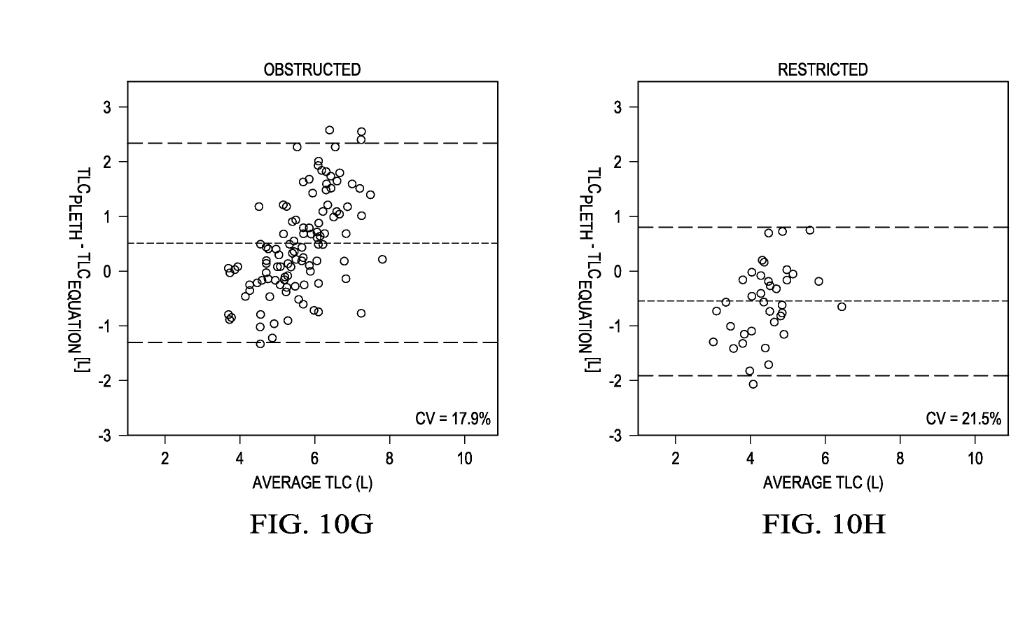

FIG. 2 illustrates another example pulmonary measurement device 200 that is configured to perform one or more processes and operations in accordance with the present disclosure. In some implementations, the device 200 may be an example of a flow interruption device (FID). In some implementations, the device 200 may be used to perform one or more operations described in the present disclosure, such as for example, one or more steps of method 600. Example data resulting from such operations performed with the device 200 is shown, for example, in FIG. 10A-10B.

Device 200 allows for controlled occlusion of the airways. In some embodiments, the device 200 may perform a rapid injection or extraction of air while measuring absolute lung volume. Device 200, in the illustrated embodiment, includes a breathing assembly 202 coupled to and in fluid communication with a container 204, which in turn is coupled to and in fluid communication with a pump 206. As illustrated, the breathing assembly 202 includes a mouthpiece 208, a flow sensor 210, a chamber 212, a pressure sensor 214, a shutter 216, and a shutter 218.

In some aspects, if shutter 218 is at its closed state, the device 200 may operate as a common interrupter device, with appropriate uses. For example, in some aspects, the shutter 218 may be configured to operate quietly so as not to create any reflexes or undesired responses by the subject, thereby avoiding inaccuracies of measurement. More importantly, shutter 218 may be configured to operate quickly, both in terms of its shutting speed (e.g., the time it takes for the shutter to go from an open state to a closed state and vice versa) and in terms of its shutting duration (e.g., the period of time for which the shutter is closed). The shutting speed is in some embodiments less than 10 ms, preferably less than 5 ms, and more preferably less than 2 ms. The shutting duration is in some embodiments less than 2 seconds and preferably less than 100 ms. This fast paced shutting speed and shutting duration may provide more accurate and reliable measurements of ALV. The high speed operation of shutter 218 and high rate of data acquisition may result from the typical response time of the lungs to abrupt occlusion of the airways while breathing. The response times of the thermodynamic and elastic properties of the lungs of a human being are in the order of a few ms to hundreds of ms, and accurate recording of the details of the response of the lungs to such abrupt occlusion is essential for accurate calculation of the internal volume of the lungs.

As illustrated, a pressure sensor 220 is mounted on a top portion of the container 204. In some embodiments, the device 200 may also include a user interface and a control module. The control module 230, generally, may include a microprocessor-based controller that is communicably coupled to, as shown, to receive measurements from the flow sensor 210 and the pressure sensor 214. The control module 230 may also be communicably coupled to the shutters 216/218 to operatively control their openings and/or closings as described below.

As illustrated, the mouthpiece 208 facilitates fluid communication between an airways (e.g., lungs) of a subject and the chamber 212 and/or container 204. For example, in some embodiments, the mouthpiece 208 may limit movement of the subject's cheeks, thereby decreasing the responsiveness thereof to the airway occlusion events.

As illustrated, shutter 216 is constructed at an end of the breathing assembly 202 opposite the mouthpiece 208. Between the shutter 216 and the mouthpiece 208, and in fluid communication with the shutter 216 and the mouthpiece 208, are the flow sensor 210 and the chamber 212. In such a manner, the shutter 216 may regulate the passage of air flux in the chamber 212.

The pressure sensor 214, in the illustrated configuration of the device 200, is positioned between the shutter 216 and the mouthpiece 208 and within the chamber 212. The pressure sensor 220, in the illustrated configuration of the device 200, is positioned to measure pressure within the container 204, e.g., on an opposite side of the shutter 218 compared to the chamber 212. The pressure sensors 214 and 220 may be any pressure measurement component, such as manometer or sensor for the measurement of absolute pressure. The pressure sensors 214 and 220 may be fabricated for example from a respiratory airflow resistive means and a differential pressure manometer, or alternatively from a Pitot tube and a differential pressure manometer.

The flow sensor 210, such as a mass respiratory airflow sensor, is positioned between the mouthpiece 208 and the chamber 212. The flow sensor 210 may be any flow sensor, such as a hot wire mass respiratory airflow sensor. In some embodiments, the respiratory airflow sensor 210 and the pressure sensor 214 may be combined in a single sensor.

The container 204 is connected to the chamber 212 with a T tube and the shutter 218 is positioned between the T tube and container 204. In some aspects, the container 204 may be made of a rigid or elastic material. In some aspects the container 204 is thermally insulated. In some aspects container 204 is an isothermal container, such as, by filling the container 204 with highly thermally conductive material, for example, copper wool.

As illustrated, the T tube may be closed to fluid communication between the chamber 212 and the container 204 by the shutter 218 (e.g., automatically and/or manually). In some embodiments, the pump 206 extracts or injects air into container 204 while the shutter 218 is closed, thereby creating a positive or negative pressure difference between the chamber 212 and the container 204.

The pump 206 may propel air away from the chamber 212, thereby to induce expiration or resist inspiration in the subject (e.g., through the mouthpiece 208). Optionally, the pump 206 may propel air into the chamber 212, and thereby induce inspiration or resist expiration in the subject. Optionally, the pump 206 may be designed to pump air in an oscillating manner thereby producing periodic movement of air in and out of chamber 212.

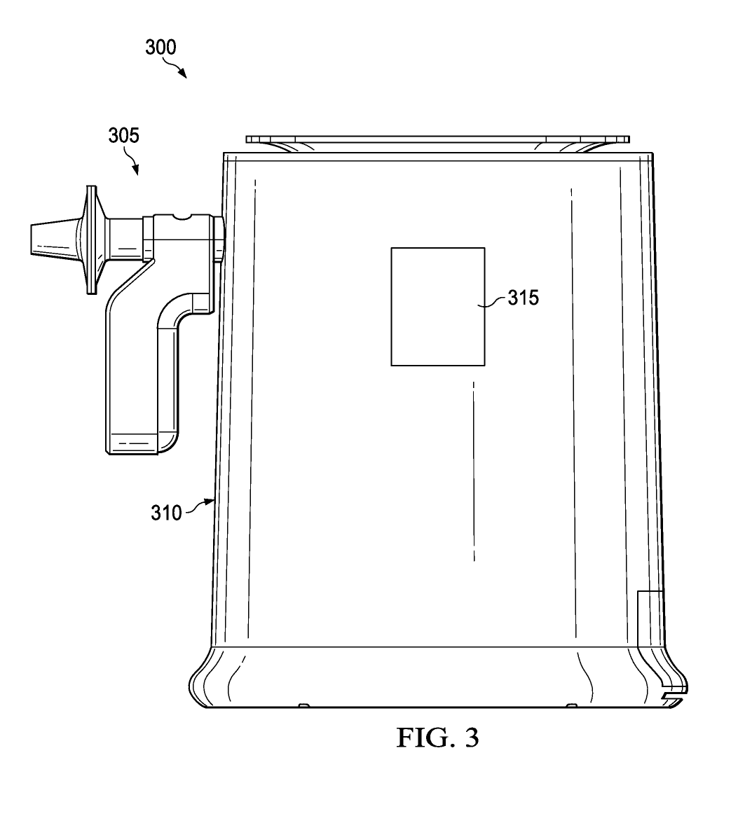

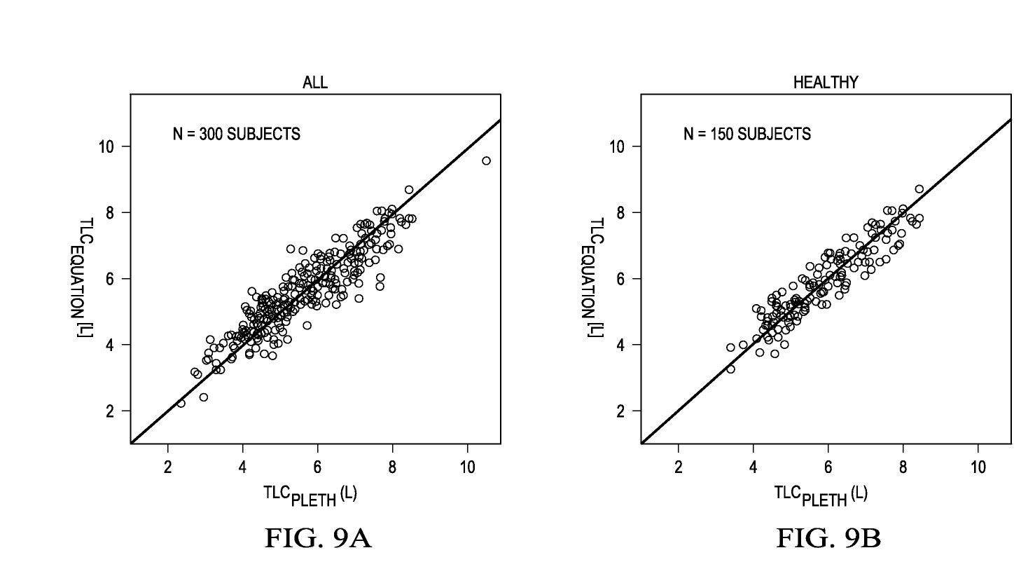

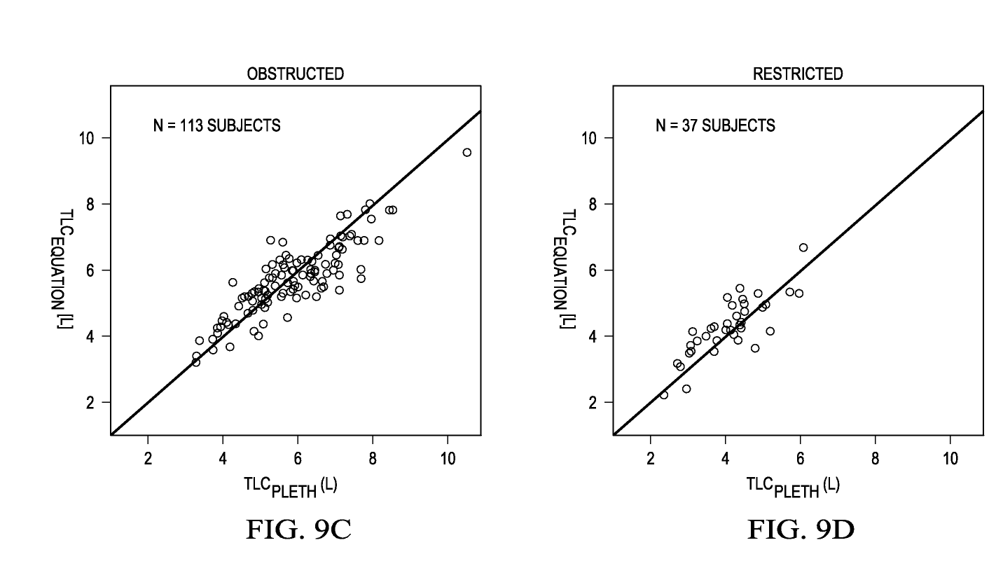

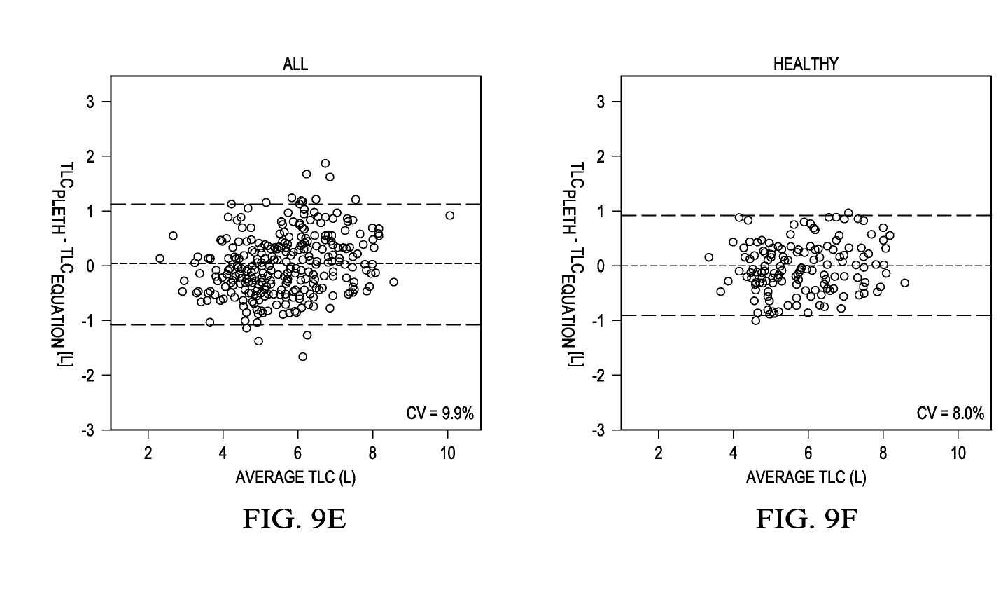

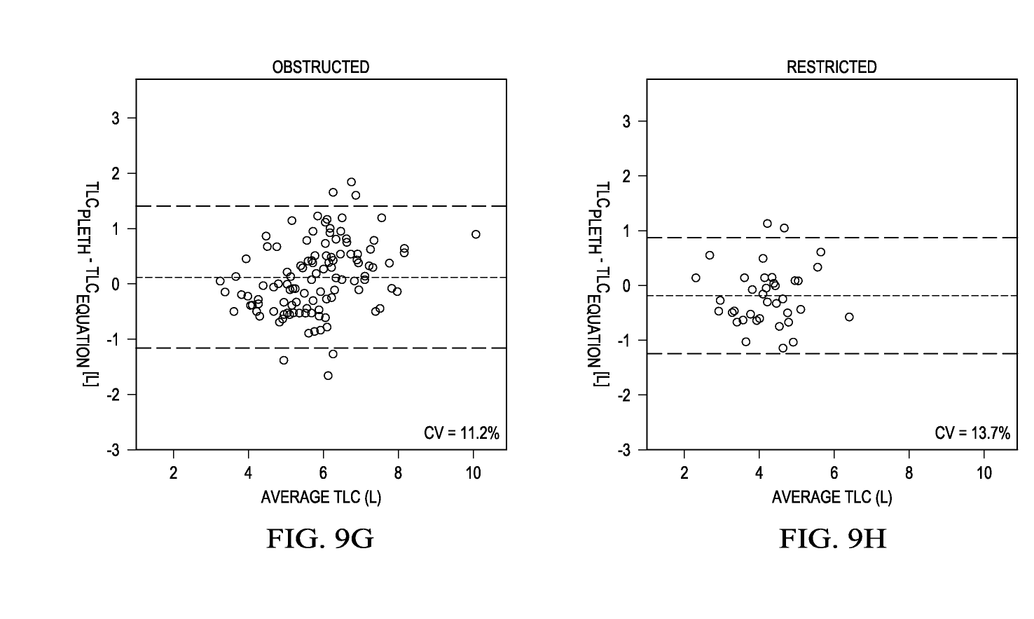

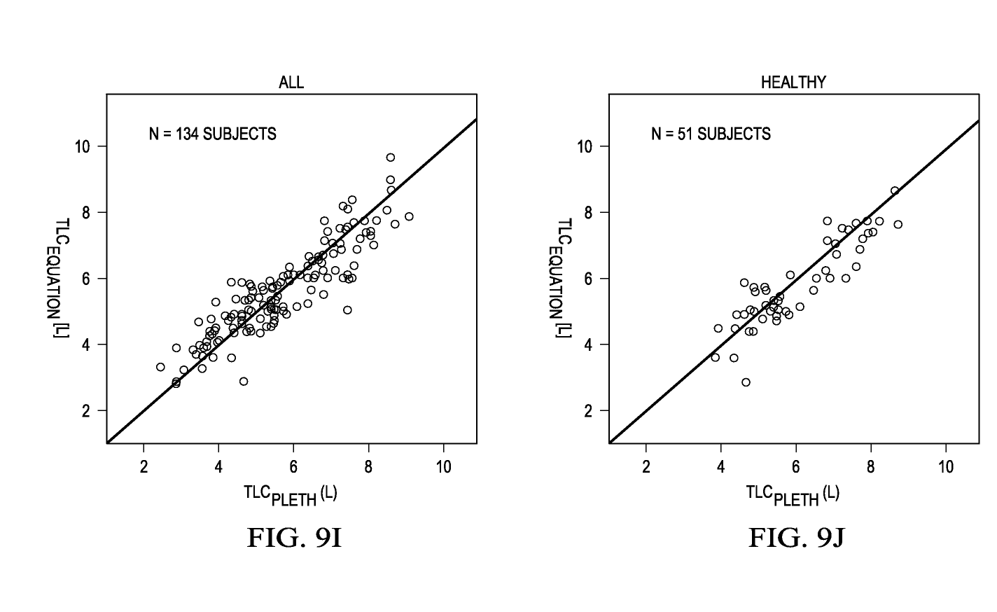

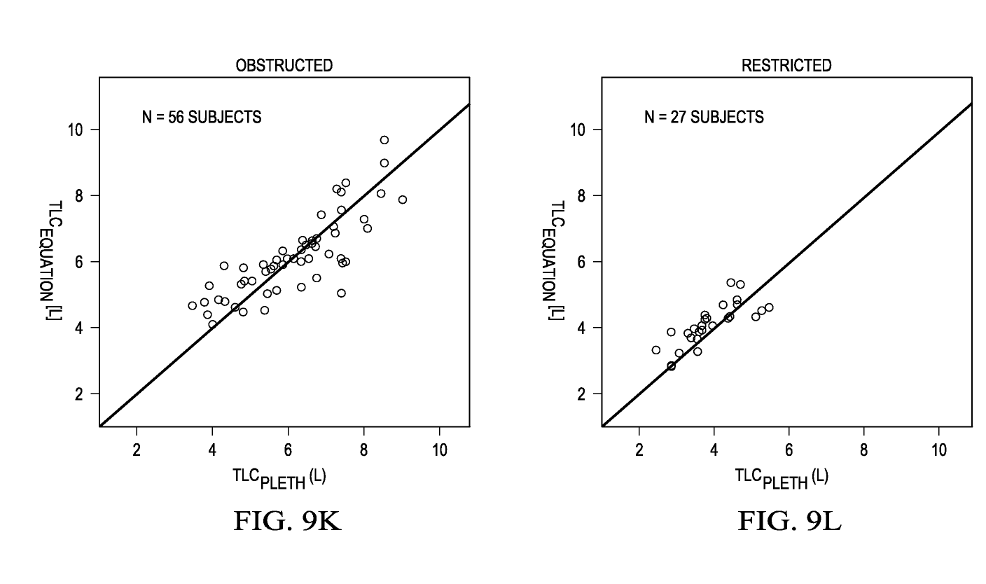

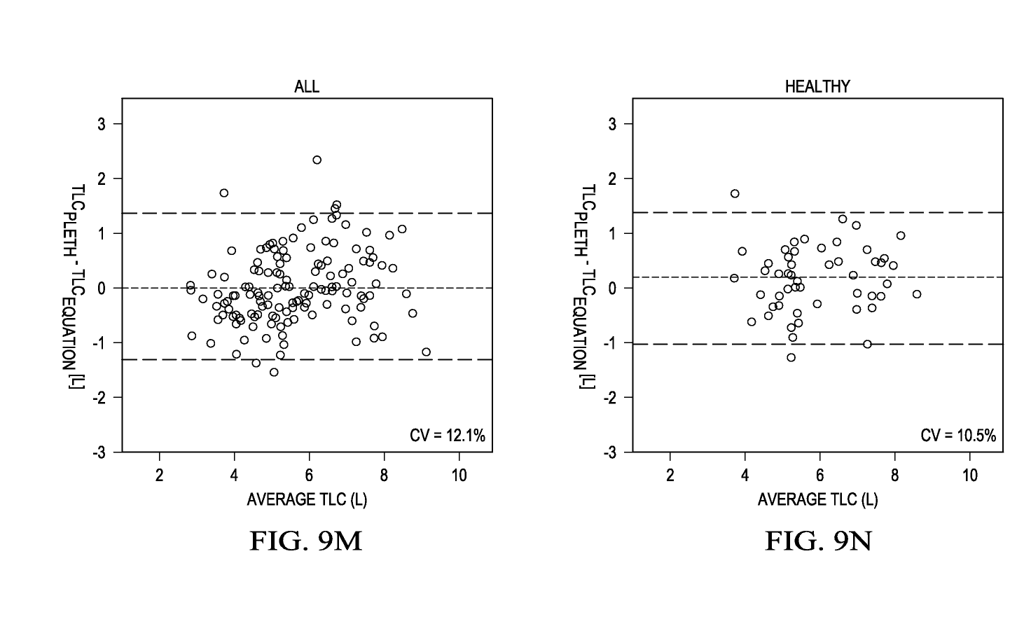

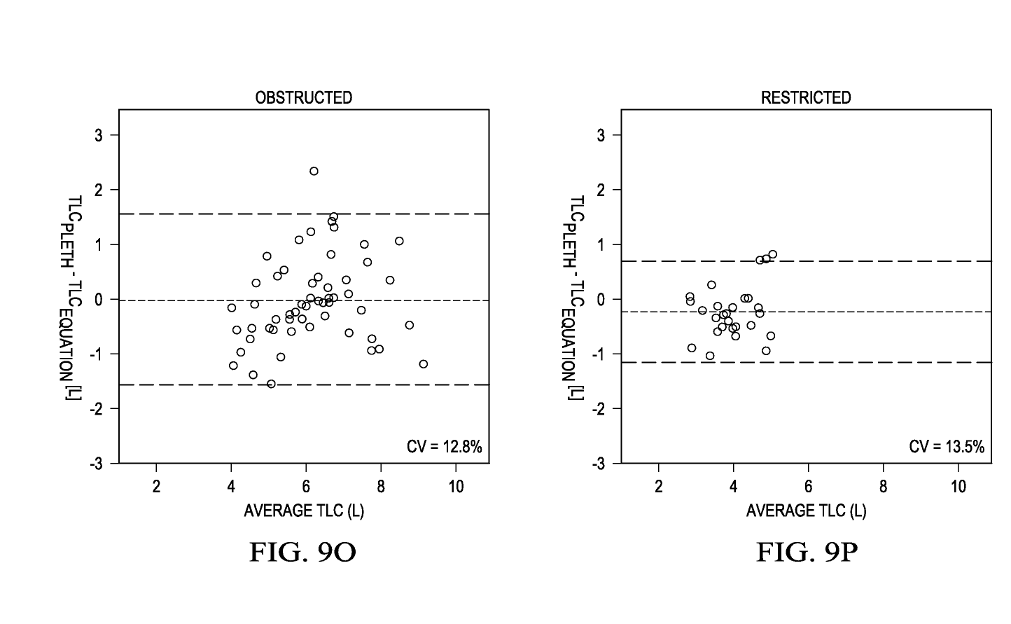

FIG. 3 illustrates an example pulmonary measurement device 300 that is configured to perform one or more processes and operations in accordance with the present disclosure. In some implementations, the device 300 may be a combination of handheld spirometer (e.g., device 100) and a flow interruption device (FID) (e.g., device 200). In some implementations, the device 300 may be used to perform one or more operations described in the present disclosure, such as for example, one or more steps of method 600. Example data resulting from such operations performed with the device 300 is shown, for example, in FIGS. 9A-9D.

Pulmonary measurement device 300, in the illustrated embodiment, includes a spirometer 305, a FID 310, and a controller 315. In some aspects, the spirometer 305 may be substantially similar (e.g., in structure and/or function) to the spirometer shown as example device 100 in FIG. 1. The FID 310, in some aspects, may be substantially similar to the FID shown as example device 200 in FIG. 2. Other spirometers and/or FIDs may also be implemented as spirometer 305 and/or FID 310, as appropriate.

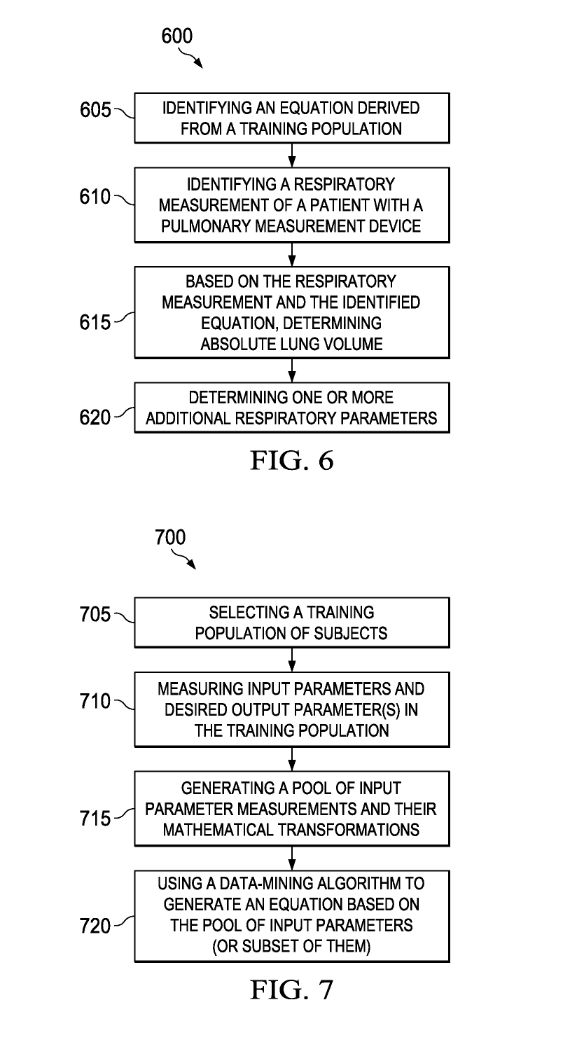

Controller 315 generally, may include a microprocessor-based controller that is communicably coupled to, as shown, to receive measurements from the spirometer 305 and the FID 310. The controller 315 may, based on measurements received from one or both of the spirometer 305 and FID 310, implement one or more equations (e.g., as described in FIGS. 6-8) to determine a respiratory parameter of a patient, such as, for instance, ALV. The controller 315 may also control the components (e.g., sensors, shutters, pumps) of the spirometer 305 and the FID 310 based on, for instance, stored instructions and/or commands from a user of the device 300.

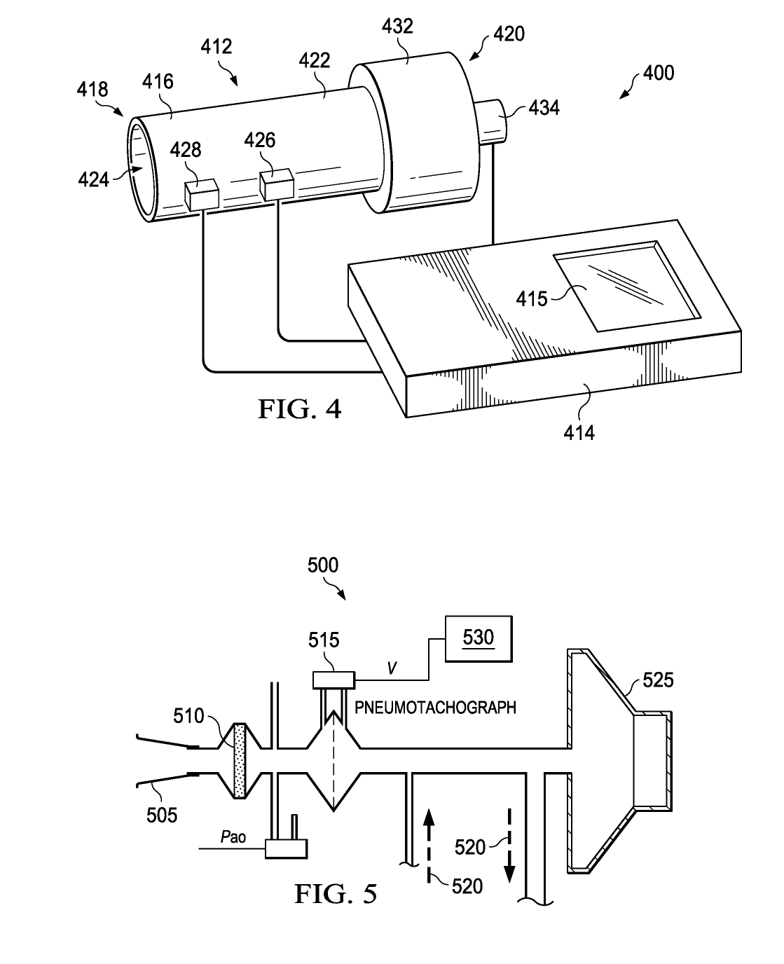

FIG. 4 illustrates an example pulmonary measurement device 400 that is configured to perform one or more processes and operations in accordance with the present disclosure. The illustrated device 400, in some aspects, may represent a container-less FID for measurement of respiration parameters. FID 400 includes a respiration module 412 and a control unit 414. Respiration module 412 is typically a hand-held device that is positionable at a mouth of a user, and is used for inhalation and/or exhalation of air for the purposes of measuring respiration parameters of the user. Respiration module 412 includes a housing 416 having a first end 418 and a second end 420, and a housing body 422 extending from first end 418 to second end 420 and defining a cavity 424 therethrough. Respiration module 412 includes a shutter assembly 432 which can open or close to allow or prevent air flow therethrough and which is controlled by a motor 434. Respiration module may be designed to introduce air flow resistance of less than 1.5 cm H₂O/Liter/sec, in accordance with ATS (American Thoracic Society) guidelines for respiratory devices.

Housing 416 may further include at least one pressure measurement component 426 and at least one air flow measurement component 428. Pressure measurement component 426 may be any suitable manometer or sensor for the measurement of absolute pressure with a data rate of at least 500 Hz; and preferably at a data rate of at least 1000 Hz. Air flow measurement component 428 may be fabricated for example from an air flow resistive means and a differential pressure manometer, or alternatively from a Pitot tube and a differential pressure manometer. The differential pressure manometer may be any suitable sensor with a data rate of at least 500 Hz; and preferably at a data rate of at least 1000 Hz. Control unit 414 is in electrical communication with pressure measurement component 426, air flow measurement component 428, and motor 434, which is used for opening and closing of a shutter mechanism.

Control unit 414 may include a converter which converts analog data received from pressure measurement component 426 and air flow measurement component 428 into digital format at a rate of at least once every 2 milliseconds (ms), and preferably at a rate at least once every 1 ms. The converter converts digital signals into commands to motor 434 for shutter assembly 432 to close and to open. Control unit 414 further includes a microprocessor which is programmed to: (a) read digital data of pressure and flow received from the converter in accordance with real-time recording, at a rate commensurate with the converter rate for each data channel and translate this digital data into pressure and flow appropriate units and store them; (b) generate signals which are sent through converter to motor 434 to command the shutter to close or to open, and (c) process above mentioned flow and pressure data in accordance with real time recording, to calculate lung volume and specifically calculate TGV, TLC and RV. The microprocessor also manages a Man-Machine Interface (MMI) that accepts operation commands from an operator and displays results. Control unit 414 may further include a display 415 for displaying the resulting values. Control unit 414 may further include a keyboard to enter subject's personal and medical information and to select desired operational modes such as shuttering duration, timing, manual versus automatic operation, calibration procedures.

Respiration module 412 may also include a mouthpiece for placement into a mouth of a user, which is attached to the shutter assembly 432. The shutter 432 may be designed specifically to minimize air displacement during opening and closing thereof. Motor 434 may be any suitable motor such as, for example, a standard solenoid. Alternatively, motor 434 may be any electronically, pneumatically, hydraulically or otherwise operated motor. Finally, a flow meter tube may be a section of respiration module 412 which is distal to shutter assembly 432, so that measurement of air flow can be taken downstream of the open or closed shutter. However, a flow meter tube may also be positioned adjacent to pressure measurement component 426.

Control unit 414 may also, based on measurements received from one or both of the pressure measurement component 426 and the air flow measurement component 428, implement one or more equations (e.g., as described in FIGS. 6-8) to determine a respiratory parameter of a patient, such as, for instance, ALV.

FIG. 5 illustrates an example pulmonary measurement device 500 that is configured to perform one or more processes and operations in accordance with the present disclosure. FIG. 5 illustrates an example flow oscillation device (FOT) 500. The FOT device 500 may contain a mouthpiece 505 coupled to a filter 510 through a flowpath, a flow source 525, a measurement module 515 (e.g., flow and/or pressure sensors), and a controller 530. In some implementations, a flow source may be any device that creates suitably fast flow fluctuations (e.g., high frequency flow oscillations). For example, the flow source 525 may be a flow actuator. In some instances, the flow source 525 can be used to generate signals including single or multiple frequencies, pseudo-random signals, impulses, and impulse trains. In some embodiments, the illustrated flow source may be a flow perturbance device, such as, for example, a loudspeaker, ventilator, or other flow actuator. Each distinct flow source which perturbs the airflow may have it owns associated waveform, for example, a pure sine wave at one frequency in the steady state or a superposition of sine waves, as two examples. Further examples may include a superposition of sine waves to produce an impulse, a frequency sweep, or a shutter.

The controller 530 of device 500 may, based on measurements received from the measurement module 515, implement one or more equations (e.g., as described in FIGS. 6-8) to determine a respiratory parameter of a patient, such as, for instance, ALV.

FIG. 6 is a flowchart of an example method 600 for calculating a desired respiratory parameter on any healthy or unhealthy subject with a pulmonary measurement device (e.g., device 100, 200, 300, 400, 500 or otherwise) that includes a mathematical equation that estimates the desired parameter. The equation, in some embodiments, that may be stored on the device and used to calculate the desired respiratory parameter, may be developed according to method 700 shown in FIG. 7, as one example.

Method 600 may begin at step 605, by identifying an equation (e.g., a single equation or a particular equation among a plurality of equations) developed from data analytics (e.g., data mining based on a training population). The equation may be stored, for instance, within executable instructions in a memory (e.g., volatile or non-volatile) that is communicably coupled to or part of a pulmonary measurement device (e.g., device 100, 200, 300, 400, 500 or other device in accordance with the present disclosure). In some aspects, the equation may be determined based on a data analytics approach using clinical data gathered (e.g., estimated with the device or directly measured by other techniques, such as whole body plethysmography or otherwise) from a training population. In some implementations, the equation is derived earlier (and possibly significantly earlier) in time relative to the implementation of step 605. The equation may be a linear or non-linear equation and may, in some examples, derived from a regression analysis.

A training population may include all healthy subjects, all unhealthy subjects and/or a mix of healthy and unhealthy subjects. In some aspects, a healthy subject may be a person that exhibits no or clinically insignificant (e.g., immeasurable) respiratory system restriction and/or obstruction. In some aspects, an unhealthy subject may be a person that exhibits clinically significant (e.g., measurable) respiratory system restriction and/or obstruction. In some aspects, an unhealthy subject may have one or more clinically-diagnosed or undiagnosed respiratory diseases. In some aspects, an unhealthy subject may demonstrate a qualitative indicator of respiratory health that includes a diagnosis of obstructive respiratory disease, restrictive respiratory disease, mixed defect, pulmonary vascular disorder, chest wall disorder, neuromuscular disorder, interstitial lung disease, pneumonitis, asthma, chronic bronchitis, and/or emphysema.

Step 610 may be implemented by identifying (e.g., from a previous or real-time measurement), or performing a respiratory measurement of a patient with the pulmonary measurement device. The respiratory measurement may be taken with the device in step 610, or may have been taken by the device prior to step 610. In some aspects, the respiratory measurement may be used as an input parameter to the equation. In some aspects, the respiratory measurement, as an input parameter, may be related to respiratory function, respiratory mechanics, or respiratory health. For instance, an airway opening pressure, a derivative of the airway opening pressure, an integral of the airway opening pressure, an airway opening flowrate, a derivative of the airway opening flowrate, an integral of the airway opening flowrate, a parameter derivable from forced spirometry, a parameter derivable from slow spirometry, a mechanical impedance, a parameter derivable from forced oscillations, a parameter derivable from impulse oscillometry, a time constant of a pressure decay or rise, or a time constant of a flowrate decay or rise, are all example respiratory measurements. As used herein, the terms “airway opening” and “mouth” are synonymous. In some aspects, therefore, the device may directly measure the respiratory measurement while in some aspects, the respiratory measurement may be derived from a direct measurement from the device.

Step 615 may be implemented by determining an absolute lung volume of any patient (e.g., healthy or un-healthy) based on the respiratory measurement and the equation identified in step 605. In one example, the appropriate equation may be implemented in software, hardware, and/or a combination thereof in a pulmonary measurement device (such as the examples described herein) such that when the input parameters are measured, the desired output respiratory parameter can be calculated automatically and instantaneously by the device. In some embodiments, the pulmonary measurement device may display the calculated respiratory parameter as well as store the values in memory.

Determining other respiratory parameters may also be performed in step 620. For example, based on the determination of absolute lung volume, or based on the respiratory measurement and the equation, one or more of TLC, FRC, TGV, RV, diffusing capacity of the lung for carbon monoxide (D_{LCO}), airway resistance, or lung tissue compliance may be determined Once such respiratory parameters (including absolute lung volume) are determined, they may be presented to the patient or other subject through the pulmonary measurement device. In some aspects, a particular equation that is initially identified or selected may be used to estimate or determine a particular respiratory parameter, such as, for example, absolute lung volume. Another particular, distinct equation may be selected or identified, in step 620, in order to determine or estimate one or more other respiratory parameters, such as those mentioned above (e.g., one or more of TLC, FRC, TGV, RV, D_{LCO}, airway resistance, or lung tissue compliance). In some aspects, each particular, distinct equation of a plurality of equations may be selected to determine or estimate a particular, distinct respiratory parameter.

Furthermore, the respiratory parameter determined in step 620 from the respiratory measurements may be a diagnosis or any qualitative measure of respiratory health (e.g., health, obstructive respiratory disease, restrictive respiratory disease, mixed defect, pulmonary vascular disorder, chest wall disorder, neuromuscular disorder, interstitial lung disease, pneumonitis, asthma, chronic bronchitis, emphysema).

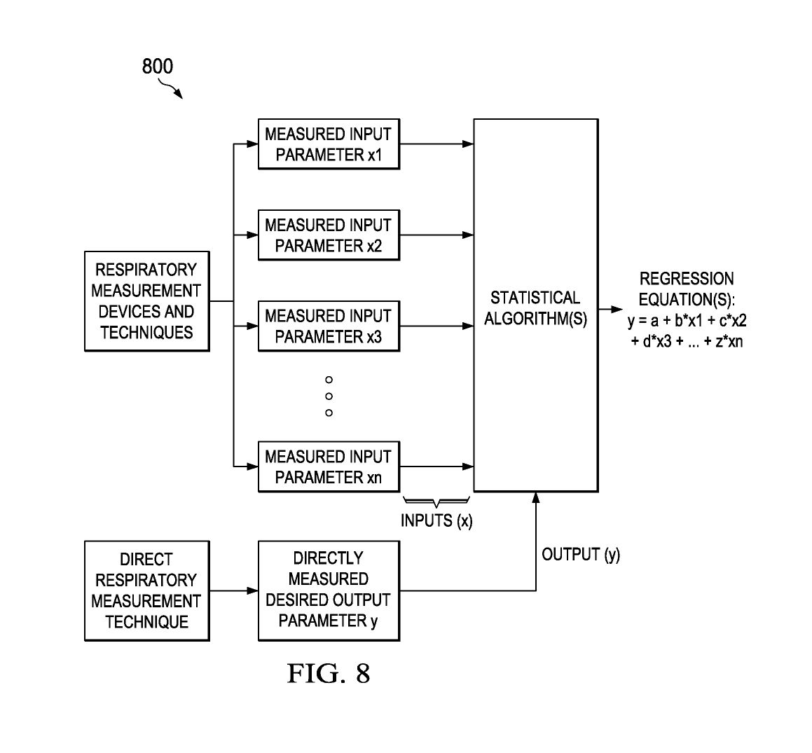

Method 600 may include one or more additional steps as well. For example, in some aspects, the equation may be updated or changed from time to time (e.g., periodically, randomly, or otherwise). For example, a training population of subjects from which the equation may be derived (e.g., according to FIGS. 7-8) may increase over time as new subjects are tested. As new subjects are tested, the equation may be updated, improved, or otherwise changed to account for the additional data. In some aspects, techniques as adapted from machine learning, artificial intelligence, and/or data mining can be used to update the equation as the number of subjects of the training population increase. This updating can occur at set intervals of time (e.g., weekly, monthly, yearly, etc.), at set increases of subjects (e.g., after each additional 10, 50, 100, subjects are added to the training population, or other interval), or when so desired. In operation in a clinical setting, the pulmonary measurement device can be used routinely to measure patients and this new patient data can also serve to further refine the mathematical equation. The mathematical equation may thus be unique to each device and tailored to the given clinical center and their patient population. It should be appreciated that there are several acceptable methods to update the mathematical equation, including via wired or wireless internet connections of the pulmonary measurement device for remote updating.

It should be appreciated that Method 600 may take place in any setting, including an intensive care unit, a pulmonary function testing laboratory, a physician's office including pulmonologists and primary care physicians, community/work screenings, and in the home setting.

FIG. 7 is a flowchart of an example process 700 for generating a mathematical equation or equations that estimates (e.g., calculates) a desired output parameter (e.g., a respiratory parameter). The mathematical equation(s) can be used during process 800 to calculate the output parameter of a patient. Example output parameters that may be estimated using such equations include TLC, TGV, FRC, RV, D_{LCO}, airway resistance, and lung tissue compliance. Example output parameters may also be qualitative indicators of respiratory health, including diagnoses of health, obstructive respiratory disease, restrictive respiratory disease, mixed defect, pulmonary vascular disorder, chest wall disorder, neuromuscular disorder, interstitial lung disease, pneumonitis, asthma, chronic bronchitis, and emphysema. An automated diagnosis of respiratory disease may be accomplished through a data analytics cluster analysis approach or similar approaches. These methods may also be used to calculate any other respiratory parameters that cannot be derived entirely from the physical principles of the measuring devices.

A training population of subjects is selected (705) on which to measure one or more input parameters (e.g., input parameters related to respiratory function, respiratory mechanics, overall respiratory health, or overall general health such as height, weight) and the desired output parameter. The number and characteristics of subjects of the training population may be selected, for example, based on diseases associated with certain values of the desired output parameter. The training population may include healthy subjects, unhealthy subjects (e.g., subjects diagnosed by a physician as having a respiratory disease, respiratory disorder, respiratory symptoms, or any other disease), or both healthy and unhealthy subjects. The number and characteristics of subjects of the training population may also be selected based on characteristics such as sex, smoking history or other characteristics. The one or more input parameters and the desired output parameter may be used in generating the mathematical equation.

The training population of subjects is then measured for the one or more input parameters and the desired output parameter (710). The one or more input parameters are measured by one or many devices and techniques (e.g., spirometry devices, respiratory mechanics devices, other hand-held, table-top, or floor-standing respiratory function devices, anthropomorphic devices, capnography, oximetry, or other devices to assess general health), and will be later used for estimating (e.g., calculating) the output parameter in other populations. The one or more input parameters may be selected based on known correlations between the one or more input parameters and the desired output parameter. Example input parameters include FEV₁, IC, VC, and height. The one or more input parameters may be selected based on postulated relation between the one or more input parameters and the desired output parameter. Example input parameters include FEV₁/FVC, airway opening pressure, airway opening flow rate, derivatives, integrals, or any other mathematical transformation of the airway opening pressure and the airway opening flow rate, mechanical impedances (e.g., in-phase and out-of-phase components), and time constants of pressure and flow rate decays and rises. In some examples, the one or more input parameters may be selected with no known or postulated relation between the one or more input parameters and the desired output parameter.

In some examples, any one of the pulmonary measurement devices shown in FIGS. 1-5 may be used to measure respiratory input parameters, such as pressures, flowrates, and time constants. Further, a pulmonary measurement device such as one described, for example, in U.S. patent application Ser. No. 12/830,955, U.S. patent application Ser. No. 12/670,661, and U.S. patent application Ser. No. 13/808,868 (each of which is incorporated by reference in its entirety as if fully set forth herein) may be used to measure respiratory input parameters, such as pressures, flowrates, and time constants and may be used in the implementation of any of the methods or processes disclosed herein. In some examples, flow interruption devices, forced oscillation devices, or impulse oscillometry may be used to measure respiratory input parameters, such as respiratory system resistance. In other examples, anthropomorphic devices may be used to measure height, weight, or body mass index (BMI).

The desired output parameter may be measured directly using a preferred device or technique (e.g., a device or technique that is considered clinically acceptable for measuring the desired output parameter). For example, preferred techniques for measuring ALV may include body plethysmography, helium dilution, and thoracic CT imaging.

A pool of input parameters can be generated from the one or more measured input parameters (715). The pool may comprise of the measured input parameters, mathematical transformations of the measured input parameters or combinations of the measured input parameters. In some examples, the pool is generated by an algorithm performed by one or more computer processors of the computer system. The pool of input parameters is used for generating an equation that comprise the input parameters (or a subset of them), which can be used for estimating (e.g., calculating) the desired output parameter (720). The equation may be generated by using one or more automated algorithms that may be carried out by the one or more processors. In some instances, the algorithm may be one or many acceptable algorithms used in the field of data analytics (e.g., data mining). In addition, the accuracy of the generated equation may be validated using one or many acceptable methods, such as cross-validation.

In some instances, some or all of the measurements described in method 700 are not explicitly performed but instead the data or part of it is acquired or otherwise obtained from existing databases or datasets of historical data that include the desired output parameters and desired input parameters. In some instances, steps 705 and 710 to measure the training population data are performed earlier (and possibly significantly earlier) in time relative to the implementation of steps 715 and 720. For example, the training population dataset may have been previously measured, may have been acquired from public databases in which the data was previously measured, or may have been acquired from private sources in which the data was previously measured.

FIG. 8 is a block diagram 800 showing the relationship among the one or more measured input parameters, the directly measured desired output parameter, and the equation (e.g., a regression equation) generated using the example process of FIG. 5. In some examples, the equation may take the form of a linear equation (1):

y=a+b*x1+c*x2+d*x3 (1)

Parameter y is the desired output parameter. Parameters x1, x2, and x3 are measurements of input parameters, as in step 710. Parameters x1, x2, and x3 were selected by an algorithm as desired input parameters, as in step 715, to be used for estimating (e.g., calculating) the desired output parameter. Lastly, parameters a, b, c, and d are scaling and shifting constants. Any one of x1, x2, and x3 may be respiratory parameters that are representative of health or disease. For example, in equation (1), x1 and x2 may account for measurements of healthy subjects, and x3 may represent measurements that account for deviations of x1 and x2 accuracy due to respiratory disease (e.g., an increase in airway resistance from a normal value due to airway obstruction).

In some examples, either or both of the conventional pulmonary measurement devices or techniques and the preferred device or technique are capable of producing measurements that account for changes in respiratory health. For example, a device may measure input parameters related to airway resistance and tissue compliance in healthy subjects, as well as input parameters that account for changes in airway resistance and tissue compliance (e.g., relative to normal measurements) in unhealthy subjects. Either or both of the one or more input parameters and the desired output parameter may be stored in memories of the devices used to perform the measurements or may be recorded in means outside of the devices. The changes in the one or more input parameters may subsequently be used as inputs to the equation for estimating (e.g., calculating) the changes in the desired output parameter, which may be useful especially in cases (e.g., certain disease states) where the preferred device or technique is inaccurate or otherwise inadequate for measuring the desired output parameter directly.

In some examples, the equation generated using the example processes 700 and/or 800 may take on a form different than that of equation (1). For example, the generated equation may be a non-linear equation, such as equation (2):

y=a+b*(x1^{c}*x2)/x3+d*x4 (2)

where, in this example, c is an exponential factor, and x4 is an additional measurement obtained using the conventional respiratory function devices and techniques. In some examples, the equation generated using the example processes 700 and/or 800 may take on a form which is significantly different from equations (1) and (2) and more easily described in some other forms, for example Decision Trees, Bayesian Networks, or any other form. In some examples, populations may first be divided into subpopulations using a measurable classifier (e.g., anthropomorphic or spirometric), and subsequently, a separate equation is generated for each subpopulation.

Since measurements of the same output parameter may differ depending on the particular preferred device or technique used to obtain the measurement (e.g., a body plethysmograph versus helium dilution), the example processes 700 and/or 800 may be used to generate equations that are distinct to the particular preferred device or measurement. For example, separate equations may be generated for estimating (e.g., calculating) TGV where the preferred device or technique used to directly measure TGV was body plethysmography, helium dilution, or thoracic CT imaging.

Once the particular equation derived, for example, from method 700, is implemented in a particular pulmonary measurement system(s) or device(s) (or other pulmonary apparatus), the device may be used to determine a desired pulmonary output parameter (e.g., TLC, TGV, lung tissue compliance, and other parameters). For example, the device may be used to measure particular input parameters from a patient or subject (e.g., healthy or unhealthy). The input parameters may include, for example, the measurements x1, x2, and x3 as well as others. Once the device measures such input parameters, the equation implemented in the device (e.g., equation (1) or other suitable equation) may be executed as described in example process 800 to determine the output parameter, y. This process can be repeated per patient or multiple times on the same patient, for example, to determine multiple desired output parameters based on a selection on the pulmonary measurement device.

More than one equation may be implemented within the pulmonary measurement system(s) or device(s). Each equation may calculate a different output parameter. Alternatively, each equation may calculate the same output parameter but the equation may be different for different groups or classes of patients (e.g., classifier models). For instance, the equation may use anthropomorphic information for each individual patient to determine which equation to utilize to calculate the output parameter.

FIGS. 9A-9D illustrate a number of charts that show a relationship between total lung capacity (TLC_{equation}) determined on a pulmonary measurement device as shown in FIG. 3 and TLC measured using the reference device body plethysmography (TLC_{PLETH}). The illustrated charts FIGS. 9A-9B, for instance, show the results of pulmonary testing on a set of training patients (e.g., about 300 subjects) that were used to develop an equation (e.g., as described in FIGS. 7 and 8). FIGS. 9A-9B show the end results of illustrated process 700; that is, a generated equation created via a training population of subjects. The illustrated charts FIGS. 9C-9D, for instance, show the results of pulmonary testing on a set of patients in the clinical setting (e.g., about 135 subjects) that were measured on the pulmonary measurement device as shown in FIG. 3 after the equation had been developed (e.g., as described in FIG. 6). FIGS. 9C-9D show the end results of illustrated process 600; that is, a final determination of absolute lung volume (e.g., TLC) from a patient in practice.

For example, FIGS. 9A-9D illustrate scatter plots of TLC generated by an equation of input parameters measured by a device such as that shown in FIG. 3 vs. plethysmographic TLC (TLCₚₗₑₜₕ). FIGS. 9A-9D illustrates the agreement between TLC_{equation} and TLCₚₗₑₜₕ for all subjects (9A), for healthy subjects only (9B), for obstructed subjects only (9C), and for restrictive subjects only (9D). The solid line represents the unity line. The agreement between TLC_{equation} and TLCₚₗₑₜₕ is shown by the data points being both centered around the line of unity and tightly clustered around the line of unity.

FIGS. 9E-9H illustrate Bland-Altman plots that are associated, respectively, with FIGS. 9A-9D. The Bland Altman plots compare TLC_{equation} to plethysmographic TLC (TLC_{PLETH}) for all subjects (9E), healthy subjects only (9F), obstructed subjects only (9G), and restrictive subjects only (9H). The solid lines represent the mean bias while the dashed lines represent the upper and lower limits (±1.96*SD). The coefficient of variation (CV) is displayed within each plot. In the population as a whole, and in each of the subpopulation (healthy, obstructed, and restrictive), the coefficients of variations were 9.91%, 7.93%, 11.30%, and 13.70% respectively; the mean biases were small (0.01 L, −0.01 L, 0.11 L, and 0.20 L, respectively); also, there was no systematic trend of variability or bias with lung size.

FIGS. 9I-9L illustrate a number of charts that show a relationship between total lung capacity (TLC_{equation}) determined on a pulmonary measurement device as shown in FIG. 3 implementing the developed equation as shown in FIGS. 9A-9H and TLC measured using body plethysmography (TLC_{PLETH}). The illustrated charts in FIGS. 9I-9L, for instance, show the results of pulmonary testing on a set of subjects with the pulmonary measurement device of FIG. 3 according to, for instance, FIG. 8. That is, the mathematical equation as developed in FIGS. 7 and 8 is implemented to measure a prospective group of subjects as in FIG. 6.

For example, FIGS. 9I-9L illustrate scatter plots of TLC_{equation} measured by a device such as that shown in FIG. 3 vs. plethysmographic TLC (TLCₚₗₑₜₕ) for all subjects (9I), healthy subjects only (9J), obstructed subjects only (9K), and restrictive subjects only (9L). The solid line represents the unity line. The agreement between TLC_{equation} and TLCₚₗₑₜₕ is shown by the data points being both centered around the line of unity and tightly clustered around the line of unity.

FIGS. 9M-9P illustrate Bland-Altman plots that are respectively associated with FIGS. 9I-9L. The Bland Altman plots compare TLC_{equation} to plethysmographic TLC (TLC_{PLETH}) for all subjects (9M), healthy subjects only (9N), obstructed subjects only (9O), and restrictive subjects only (9P). The solid lines represent the mean bias while the dashed lines represent the upper and lower limits (±1.96*SD). The coefficient of variation (CV) is displayed within each plot.

FIGS. 10A-10H illustrate a number of charts that show a relationship between TLC generated by an equation (TLC_{equation}) of input parameters measured by a device such as that shown in FIG. 2 and TLC measured using body plethysmography (TLC_{PLETH}). FIGS. 10A-10B illustrate the agreement between TLC_{equation} and TLCₚₗₑₜₕ for all subjects (10A), for healthy subjects only (10B), for obstructed subjects only (10C), and for restrictive subjects only (10D). The solid line represents the unity line. The agreement between TLC_{equation} and TLCₚₗₑₜₕ is shown by the data points being both centered around the line of unity and tightly clustered around the line of unity. FIGS. 10E-10H illustrate Bland-Altman plots that are respectively associated with FIGS. 10A-10D. The Bland Altman plots compare TLC_{equation} to plethysmographic TLC (TLC_{PLETH}) for all subjects (10E), healthy subjects only (10F), obstructed subjects only (10G), and restrictive subjects only (10H). The solid lines represent the mean bias while the dashed lines represent the upper and lower limits (±1.96*SD). The coefficient of variation (CV) is displayed within each plot.

FIGS. 11A-11H illustrate a number of charts that show a relationship between TLC generated by an equation (TLC_{equation}) of input parameters measured by a device such as that shown in FIG. 1 and TLC measured using body plethysmography (TLC_{PLETH}). FIGS. 11A-11D illustrate the agreement between TLC_{equation} and TLCₚₗₑₜₕ for all subjects (11A), for healthy subjects only (11B), for obstructed subjects only (11C), and for restrictive subjects only (11D). The solid line represents the unity line. The agreement between TLC_{equation} and TLCₚₗₑₜₕ is shown by the data points being both centered around the line of unity and tightly clustered around the line of unity. FIGS. 11E-11H illustrate Bland-Altman plots that are respectively associated with FIGS. 11A-11D. The Bland Altman plots compare TLC_{equation} to plethysmographic TLC (TLC_{PLETH}) for all subjects (11E), healthy subjects only (11F), obstructed subjects only (11G), and restrictive subjects only (11H). The solid lines represent the mean bias while the dashed lines represent the upper and lower limits (±1.96*SD). The coefficient of variation (CV) is displayed within each plot.

Table 1 (below) shows statistical parameters characterizing the data shown in FIGS. 9A-9D and FIGS. 9E-9H. In particular, Table 1 provides the population size (N), the mean TLC_{PLETH}, the mean TLC_{equation}, the root mean square error (RMSE), and the coefficient of variation (CV).

TABLE 1

| | | Mean | Mean TLC_{equation} | RMSE | |
|---|---|---|---|---|---|
| | N | TLC_{PLETH} (L) | (L) | (L) | CV (%) |
| Healthy | 150 | 5.82 L | 5.83 L | 0.46 L | 8.0% |
| Obstructed | 113 | 5.87 L | 5.75 L | 0.66 L | 11.2% |
| Restricted | 37 | 4.10 L | 4.30 L | 0.56 L | 13.7% |
| Total | 300 | 5.63 L | 5.61 L | 0.56 L | 9.9% |

A particular equation (e.g., developed through method 700) implemented in a pulmonary measurement device (e.g., through method 600) can be implemented in digital electronic circuitry, in tangibly-embodied computer software or firmware, in computer hardware, or in combinations of one or more of them. Implementations of the equation can be implemented as one or more computer programs, e.g., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The equation implemented in the pulmonary measurement device can be executed by “data processing hardware,” including by way of example a programmable processor, a computer, or multiple processors or computers. The hardware can also be or further include special purpose logic circuitry, e.g., a central processing unit (CPU), a FPGA (field programmable gate array), or an ASIC (application-specific integrated circuit). In some implementations, the data processing apparatus and/or special purpose logic circuitry may be hardware-based and/or software-based. For example, data processing hardware may include the controller 140 (shown in FIG. 1), the controller 230 (shown in FIG. 2), the control module 315 (shown in FIG. 3), the control unit 415 (shown in FIG. 4), and/or a controller 530 (shown in FIG. 5).

The processes and logic flows implemented by the equation can be performed by one or more programmable computers executing one or more computer programs to perform functions by operating on input data and generating output. The processes and logic flows can also be performed by, and apparatus can also be implemented as, special purpose logic circuitry, e.g., a central processing unit (CPU), a FPGA (field programmable gate array), or an ASIC (application-specific integrated circuit).

Computers suitable for the execution of a computer program include, by way of example, can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices.

Computer-readable media (transitory or non-transitory, as appropriate) suitable for storing computer program instructions and data, such as instructions and data associated with the equation implemented in the pulmonary measurement device, include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The memory may store various objects or data, including caches, classes, frameworks, applications, backup data, jobs, web pages, web page templates, database tables, repositories storing business and/or dynamic information, and any other appropriate information including any parameters, variables, algorithms, instructions, rules, constraints, or references thereto. Additionally, the memory may include any other appropriate data, such as logs, policies, security or access data, reporting files, as well as others. The processor and the memory can be supplemented by, or incorporated in, special purpose logic circuitry.

To provide for interaction with a user, implementations of the subject matter described in this specification can be implemented on a pulmonary measurement device having, or connected to, a display device, e.g., a CRT (cathode ray tube), LCD (liquid crystal display), or plasma monitor, for displaying information to the user and an input device (e.g., keypad, a pointing device, or otherwise), by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

A number of embodiments have been described. Nevertheless, it will be understood that various modifications may be made. For example, an example embodiment of a method to calculate a desired respiratory parameter includes determining an equation that comprises one or more input parameters associated with a plurality of baseline respiratory measurements where some of the input respiratory measurements are used to calculate the desired respiratory parameter in a subject; measuring at least one respiratory measurement of a patient with a respiratory device; and based on the measured respiratory measurement of the patient as an input into the equation, determining the desired respiratory parameter of the patient. Example aspects combinable with the example embodiment include: the desired respiratory parameter comprises at least one of TLC, TGV, RV, D_{LCO}, airway resistance, lung tissue compliance, or FRC; the equation is determined based on an algorithmic approach using clinical data; the respiratory device is configured to obtain values of one or more respiratory parameters to be used in the equation; the training population comprises healthy subjects; the training population comprises unhealthy subjects; the unhealthy subjects have one or more respiratory diseases; the subject health condition is a blend of healthy and unhealthy; the one or more input parameters comprise parameters related to respiratory function, respiratory mechanics, or overall respiratory health; the one or more input parameters can be selected based on known correlations between the one or more input parameters and the desired respiratory parameter; the one or more input parameters can comprise an airway opening pressure, a derivative of the airway opening pressure, an integral of the airway opening pressure, an airway opening flowrate, a derivative of the airway opening flowrate, an integral of the airway opening flowrate, a mechanical impedance, a time constant of a pressure decay or rise, and a time constant of a flowrate decay or rise; the one or more input parameters are measured using one or more of a spirometry device or a respiratory mechanics device; the desired respiratory health parameter is measured directly; the desired respiratory parameter is measured using a preferred device or technique; the preferred device or technique comprises body plethysmography, helium dilution, or thoracic CT imaging; the equation comprises a linear equation; the equation comprises a non-linear equation; the equation is generated using an algorithmic approach; and/or the equation comprises one or more input parameters.

Another example embodiment of a method for estimating a respiratory parameter includes determining at least one measurement of a respiratory parameter of a subject; inputting the measurement into an equation developed based on a plurality of historical measurements of the respiratory parameter; and outputting a respiratory parameter from the equation, the respiratory parameter comprising an estimate of TLC, FRC, or TGV.

Another example embodiment of a method of estimating a respiratory parameter of a human subject includes (1) taking a direct measurement of a respiratory parameter in a plurality of test subjects; (2) taking a measurement of one or more input parameters of the plurality of test subjects, each of the input parameters associated with the respiratory parameter; (3) determining, with the measurements of the desired respiratory parameter and the measurements of one or more input parameters, an equation that comprises at least a portion of the input parameters or subset of them as inputs and the pulmonary parameter as an output; (4) taking a measurement of one or more parameters of the human subject; and (5) based on the measurement of one or more parameters of the human subject and the equation, estimating a value of the respiratory parameter of the human subject. Example aspects combinable with the example embodiment include: step (1) is performed by a whole body plethysmography technique, a helium dilution technique, or a thoracic CT imaging technique, a nitrogen washout, a nitrogen recovery, or a chest radiography; steps (2) and (4) are performed with a respiratory device, a forced oscillation or impulse oscillometry technique or with an anthropomorphic device; the respiratory device comprises a device as described in one of U.S. patent application Ser. No. 12/830,955, U.S. patent application Ser. No. 12/670,661, or U.S. patent application Ser. No. 13/808,868; the input parameters comprise relative lung volumes or lung flow rates such as FEV₁, FEV₁/FVC, IC, or VC; some of the input parameters can comprise respiratory pressure, respiratory flow rates, or other respiratory dynamics; some of the input parameters can comprise respiratory mechanics values such as Rᵣₛ or Eᵣₛ; some of the input parameters can comprise anthropomorphic information that include one or more of patient sex, patient height, patient weight, or patient body mass index; and/or the respiratory parameter comprises at least one of TLC, TGV, RV, or FRC.

Another example embodiment of a method of generating an equation that estimates a desired respiratory parameter includes selecting a training population of subjects on which to measure one or more input parameters and the desired respiratory parameter; measuring the training population for the one or more input parameters and the desired respiratory parameter; generating a pool of input parameters from the one or more input parameters by means of mathematical transformations; and generating an equation using measurements of a subset of input parameters and measurements that can be used for estimating of the desired respiratory parameter.

Various combinations of the components described herein may be provided for embodiments of a similar apparatus. Accordingly, other embodiments are within the scope of the present disclosure.

What is claimed is:

1. A pulmonary measurement system, comprising:
a pulmonary measurement device that comprises:
a mouthpiece that comprises an airflow path; and
a sensor positioned in the airflow path; and
a controller communicably coupled to the sensor, the controller comprising a processor and instructions stored in memory, the controller operable to execute the instructions with the processor to perform operations comprising:
identifying a measurement from the sensor;
identifying a particular equation stored in the memory, the particular equation developed using data analytics and comprising an input parameter that is based on the identified measurement; and
based on the identified measurement and the particular equation, determining a value of absolute lung volume.

2. The pulmonary measurement system of claim 1, wherein the sensor comprises at least one of an airflow sensor or a pressure sensor.

3. The pulmonary measurement system of claim 1, wherein the controller is operable to execute the instructions with the processor to perform further operations comprising:
determining the input parameter to the particular equation based on the measurement; and
calculating the value of absolute lung volume based on the input parameter.

4. The pulmonary measurement system of claim 1, wherein the input parameter comprises a parameter related to respiratory function, respiratory mechanics, respiratory health, or general health.

5. The pulmonary measurement system of claim 1, wherein the input parameter comprises at least one of an airway opening pressure, a derivative of the airway opening pressure, an integral of the airway opening pressure, an airway opening flowrate, a derivative of the airway opening flowrate, an integral of the airway opening flowrate, a parameter derivable from forced spirometry, a parameter derivable from slow spirometry, a mechanical impedance, a parameter derivable from forced oscillations, a parameter derivable from impulse oscillometry, a time constant of a pressure decay or rise, or a time constant of a flowrate decay or rise.

6. The pulmonary measurement system of claim 1, wherein the pulmonary measurement device comprises one of:
(a) a spirometer;
(b) a forced oscillation device;
(c) an advanced flow interruption device;
(d) a flow interruption device;
(e) a combination spirometer-flow interruption device; or
(f) a combination device of two or more of (a)-(e).

7. The pulmonary measurement system of claim 1, further comprising a handheld housing that at least partially encloses or couples to the pulmonary measurement device and the controller.

8. The pulmonary measurement system of claim 1, wherein identifying a particular equation comprises identifying the particular equation from a plurality of equations that are stored in the memory.

9. The pulmonary measurement system of claim 8, wherein the controller is operable to execute the instructions with the processor to perform further operations comprising:
identifying a second particular equation of the plurality of equations that are stored in the memory, the second particular equation developed using data analytics and comprising a second input parameter that is based on the identified measurement; and
based on the identified measurement and the second particular equation, determining at least one of total lung capacity (TLC), functional residual capacity (FRC), thoracic gas volume (TGV), residual volume (RV), diffusing capacity of the lung for carbon monoxide (D_{LCO}), airway resistance, lung elasticity, or lung tissue compliance.

10. The pulmonary measurement system of claim 8, wherein the controller is operable to execute the instructions with the processor to perform further operations comprising:
identifying a second particular equation of the plurality of equations that are stored in the memory, the second particular equation developed using data analytics and comprising a second input parameter that is based on the identified measurement; and
based on the identified measurement and the second particular equation, determining at least one qualitative indicator of respiratory health.

11. The pulmonary measurement system of claim 10, wherein the at least one qualitative indicator of respiratory health comprises a diagnosis of: health, obstructive respiratory disease, restrictive respiratory disease, mixed defect, pulmonary vascular disorder, chest wall disorder, neuromuscular disorder, interstitial lung disease, pneumonitis, asthma, chronic bronchitis, or emphysema.

12. The pulmonary measurement system of claim 1, wherein the particular equation is derived from a training population that comprises a plurality of healthy subjects.

13. The pulmonary measurement system of claim 12, wherein the particular equation is derived from a training population that further comprises a plurality of unhealthy subjects.

14. The pulmonary measurement system of claim 13, wherein each of the plurality of unhealthy subjects has one or more respiratory diseases.

15. The pulmonary measurement system of claim 12, wherein the particular equation comprises a constant that is calculated based on a respiratory measurement technique performed on the training population.

16. The pulmonary measurement system of claim 15, wherein the respiratory measurement technique comprises at least one of body plethysmography, helium dilution, or thoracic computed tomography (CT) imaging.

17. The pulmonary measurement system of claim 12, wherein the training population comprises historical or public data.

18. The pulmonary measurement system of claim 12, wherein the training population comprises a first portion and a second portion, each of the first and second portions defined by a classifier.

19. The pulmonary measurement system of claim 18, wherein the classifier comprises an anthropomorphic or a spirometric classifier, and the controller is operable to execute the instructions with the processor to perform further operations comprising:
selecting the particular equation based, at least in part, on the classifier.

20. The pulmonary measurement system of claim 1, wherein the respiratory measurement occurs in one of an intensive care unit, a pulmonary function testing laboratory, a physician's office, a community/work screening, or a home setting.

21. The pulmonary measurement system of claim 1, wherein the particular equation comprises a linear equation or a non-linear equation.

22. The pulmonary measurement testing system of claim 1, wherein the particular equation is derived from a regression analysis.

23. The pulmonary measurement testing system of claim 1, wherein the controller is operable to execute the instructions with the processor to perform further operations comprising:
updating at least one of the plurality of equations that are stored in the memory based on at least one of a time duration or an adjustment to the data analytics.

24. The pulmonary measurement testing system of claim 23, wherein the adjustment to the data analytics comprises an increase in a number of subjects of a training population used to derive the plurality of equations.

25. A computer-implemented method to determine absolute lung volume, comprising:
identifying a respiratory measurement of a patient with a pulmonary measurement device;
identifying a particular equation that is developed using data analytics and comprises an input parameter, the input parameter based on the identified respiratory measurement; and
based on the respiratory measurement of the patient and the particular equation, determining the absolute lung volume of the patient.

26. The computer-implemented method of claim 25, wherein identifying a particular equation comprises identifying the particular equation from a plurality of equations.

27. The computer-implemented method of claim 26, further comprising:
identifying a second particular equation of the plurality of equations that is developed using data analytics and comprises a second input parameter; and
based on the respiratory measurement of the patient and the second particular equation, determining at least one of total lung capacity (TLC), functional residual capacity (FRC), thoracic gas volume (TGV), residual volume (RV), diffusing capacity of the lung for carbon monoxide (D_{LCO}), airway resistance, or lung tissue compliance.

28. The computer-implemented method of claim 25, wherein the particular equation is determined based on a training population using clinical data.

29. The computer-implemented method of claim 28, wherein the training population comprises healthy subjects and unhealthy subjects.

30. The computer-implemented method of claim 29, wherein each of the unhealthy subjects have one or more respiratory diseases.

31. The computer-implemented method of claim 28, further comprising:
generating the data analytics by measuring an absolute lung volume value of each subject of the training population using a respiratory measurement technique.

32. The computer-implemented method of claim 25, further comprising obtaining the at least one respiratory measurement with the pulmonary measurement device.

33. The computer-implemented method of claim 32, wherein the pulmonary measurement device comprises one of:
(a) a spirometer;
(b) a forced oscillation device;
(c) an advanced flow interruption device;
(d) a flow interruption device;
(e) a combination spirometer-flow interruption device; or
(f) a combination device of two or more of (a)-(e).

34. The computer-implemented method of claim 25, wherein the input parameter comprises a parameter related to respiratory function, respiratory mechanics, respiratory health, or general health.

35. The computer-implemented method of claim 25, wherein the input parameter is selected based on a known correlation between the input parameter and absolute lung volume.

36. The computer-implemented method of claim 25, wherein the input parameter comprises at least one of an airway opening pressure, a derivative of the airway opening pressure, an integral of the airway opening pressure, an airway opening flowrate, a derivative of the airway opening flowrate, an integral of the airway opening flowrate, a parameter derivable from forced spirometry, a parameter derivable from slow spirometry, a mechanical impedance, a parameter derivable from forced oscillations, a parameter derivable from impulse oscillometry, a time constant of a pressure decay or rise, or a time constant of a flowrate decay or rise.

37. The computer-implemented method of claim 31, wherein the respiratory measurement technique comprises body plethysmography, helium dilution, or thoracic computed tomography (CT) imaging.

38. The computer-implemented method of claim 25, wherein the particular equation comprises a linear equation.

39. A method of estimating a respiratory parameter of a human subject, comprising:
taking a direct measurement of a respiratory parameter in a plurality of test subjects, the plurality of test subjects comprising healthy subjects and unhealthy subjects;
taking a measurement of one or more input parameters of the plurality of test subjects; and
determining, with the direct measurements of the respiratory parameter and the measurements of one or more input parameters, an equation that comprises at least a portion of the input parameters as inputs and the respiratory parameter as an output.

40. The method of claim 39, wherein each of input parameters is associated with the respiratory parameter.

41. The method of claim 39, wherein taking a direct measurement of a respiratory parameter in a plurality of test subjects is performed with at least one of:
a whole body plethysmography technique, a helium dilution technique, a thoracic computed tomography (CT) imaging technique, a nitrogen washout, a nitrogen recovery, or a chest radiography.

42. The method of claim 39, wherein taking a measurement of one or more input parameters of the plurality of test subjects is performed with at least one of:
a pulmonary measurement device, a spirometer, a flow interruption device, an advanced flow interruption device, a forced oscillation or impulse oscillometry technique, or an anthropomorphic device.

43. The method of claim 39, wherein at least one of the one or more input parameters comprises a relative lung volume or a lung flow rate.

44. The method of claim 43, wherein the relative lung volume comprises at least one of: forced expiratory volume in one second (FEV₁), a ratio of forced expiratory volume in one second to forced vital capacity (FEV₁/FVC), inspiratory capacity (IC), or vital capacity (VC).

45. The method of claim 39, wherein at least one of the one or more input parameters comprises at least one of an airway opening pressure, a derivative of the airway opening pressure, an integral of the airway opening pressure, an airway opening flowrate, a derivative of the airway opening flowrate, an integral of the airway opening flowrate, a parameter derivable from forced spirometry, a parameter derivable from slow spirometry, a mechanical impedance, a parameter derivable from forced oscillations, a parameter derivable from impulse oscillometry, a time constant of a pressure decay or rise, or a time constant of a flowrate decay or rise.

46. The method of claim 39, wherein at least one of the one or more input parameters comprises a respiratory mechanics value comprising respiratory system resistance (Rᵣₛ) or respiratory system elastance (Eᵣₛ).

47. The method of claim 39, wherein at least one of the one or more input parameters comprises anthropomorphic information that includes one or more of patient sex, patient height, patient weight, or patient body mass index.

48. The method of claim 39, wherein the respiratory parameter comprises at least one of: total lung capacity (TLC), thoracic gas volume (TGV), residual volume (RV), or functional residual capacity (FRC).

49. The method of claim 39, further comprising:
taking a measurement of one or more input parameters of a human subject with a pulmonary measurement device; and
based on the measurement of one or more input parameters of the human subject and the equation, estimating a value of the respiratory parameter of the human subject with the pulmonary measurement device.
